(19) United States
(12) Patent Application Publication
(10) Pub. No.: US 2005/0255039 A1
(43) Pub. Date: Nov. 17, 2005

(54) Gel injection treatment of breast, fibroids & endometrial ablation

(75) Inventor: Ashvin Desai, San Jose, CA (US)

Correspondence Address: FISH & RICHARDSON P.C., PO BOX 1022, MINNEAPOLIS, MN, 55440-1022, US

(73) Assignee: Pro Surg, Inc., a California Corporation

(21) Appl. No.: 11/145,677

(22) Filed: Jun. 6, 2005

Related U.S. Application Data
(62) Division of application No. 10/274,436, filed on Oct. 17, 2002.
Application No. 10/274,436 is a continuation-in-part of application No. 10/193,721, filed on Jul. 10, 2002.
Application No. 10/274,436 is a continuation-in-part of application No. 09/715,853, filed on Nov. 17, 2000.
Application No. 09/715,853 is a continuation-in-part of application No. 09/510,537, filed on Feb. 22, 2000, now Pat. No. 6,461,296.
Application No. 09/510,537 is a continuation-in-part of application No. 09/105,896, filed on Jun. 26, 1998, now Pat. No. 6,231,591.
(60) Provisional application No. 60/383,015, filed on May 23, 2002.

(51) Int. Cl. A61M 36/14; A61K 51/00; A61K 38/17; A61K 31/716; A61K 38/43; A61K 39/21; A61K 31/19; A61K 31/137

(52) U.S. Cl. 424/1.11; 424/488; 514/57; 514/649; 514/44; 424/9.6; 424/9.3; 424/725; 514/2; 424/208.1; 424/93.2; 514/724; 514/557; 424/94.1; 604/500

(57) ABSTRACT

A method and apparatus for treatment of diseases of a breast, fallopian tube and female reproductive organs by injecting/delivering a treatment substance/chemo-gel, directly into the target tissue of the body organ, and thereby leaving the remaining body organs relatively unaffected. Specific formulations and composition of treatment substances are provided. The typical treatment substance/chemo-gel formulations contain at least two principle components including an active treatment (therapy) substance, and an inactive binding (carrier) substance for thickening the treatment substance. Specific substance formulations are provided, including a range of recommended dosage levels for optimum treatment of a specific disease of a female reproductive organ. Injection/delivery apparatus and methods, and apparatus for guiding its placement are also provided.

### RELATED CASES

The present application is a continuation-in-part of co-pending U.S. patent application Ser. No. 10/193,721 filed on Jul. 10, 2002 (which claims priority from U.S. Provisional Patent Application 60/383,015 filed May 23, 2002), which is a continuation-in-part of U.S. patent application Ser. No. 09/715,853 filed Nov. 17, 2000 which is a continuation-in-part of U.S. Patent Application U.S. patent application Ser. No. 09/510,537 filed Feb. 22, 2000, which is a continuation-in-part of U.S. patent application Ser. No. 09/105,896 filed Jun. 26, 1998, which is a continuation-in-part of U.S. patent application Ser. No. 08/639,199 filed Apr. 26, 1996, which is a continuation-in-part of U.S. patent application Ser. No. 08/259,712 filed Jun. 14, 1994, which is a continuation-in-part of U.S. patent application Ser. No. 08/025,003 filed Mar. 2, 1993, which is a continuation-in-part of U.S. patent application Ser. No. 07/779,108 filed Oct. 18, 1991. The contents of each of these applications is incorporated in this application by reference.

### 1. BACKGROUND OF THE INVENTION

Field of the Invention

The present invention relates generally to methods and apparatus for treating diseases of body organs including the breast, fibroids and uterus, uterine cavity, and more specifically to treatment involving the injection of a treatment substance of a specific composition and formulation into a body organ for treatment of particular disease conditions including breast tumors, cysts, fibroadenoma, breast cancer, uterine fibroids, cancer of reproductive organs, and for treatment of menorrhagia including endometrial ablation.

3. Description of the Prior Art

(A) Uterine Fibroid Ablation:

A variety of minimally invasive surgical approaches and injection treatments are currently known to be of benefit in treating disease conditions in body organs including the breast, fibroids and uterus, and for treating other female reproductive organ disorders. One out of every four or five women over the age of 35 have uterine fibroids. These are non-cancerous tumors of the uterus that appear during childbearing years. Also called myomas, fibromyomas or leiomyomas, fibroids can appear on the inside or outside lining of the uterus, or within its muscular wall. They usually develop from a single smooth muscle cell that continues to grow. Fibroids remain the number one reason for hysterectomy with 150,000 to 175,000 operations carried out each year because of fibroids.

Currently, there are a number of treatment options available for abnormal bleeding caused by uterine fibroids.
(i) Non-Steroidal Anti-Inflammatory Drugs (NSAIDs, e.g., Ibuprofen)
(ii) Vitamin and/or herbal remedies
(iii) Dilation and Curettage (D&C)
(iv) Hormonal therapy (e.g., birth control pills)

Hysterectomy is certainly a treatment option.

There are a variety of alternate treatment options for benign fibroids which allow women to retain their uterus. Many women choose to do nothing and simply treat the symptoms since fibroids often shrink in size and become asymptomatic as a woman goes through menopause. The average age of menopause is 51. Myomectomy is a type of surgery that removes the fibroid without removing the uterus. For younger women over the age of 35, this procedure may provide adequate relief until the age of menopause when fibroids shrink naturally due to a decline in hormones.

(B) Endometrial Ablation:

Uterine fibroid embolization (UFE, also known as uterine artery embolization UAE) is a minimally-invasive, non-surgical procedure performed by an interventional radiologist (IR). This procedure involves placing a catheter into the artery and guiding it to the uterus. Small particles are then injected into the artery. The particles block the blood supply feeding the fibroids. Within minutes after the procedure the fibroids begin dying.

Myolysis involves surgical instruments that are inserted through a laparoscopic incision in the abdomen (usually the navel) and a high frequency RF electrical current is sent to the fibroid. The electrical current causes the blood vessels to vaso-constrict and this cuts off the blood flow to the fibroids. Myolysis is only performed on subserosal fibroids that fit a certain size range.

Menorrhagia is a medical condition in women which manifests symptoms including excessive and difficult to control bleeding of the endometrial layer of the uterus. The endometrium is usually thought of as the inner lining of the uterus to which an embryo normally attaches and, typically excludes the portion of the uterine inner lining forming the cervix. The symptoms of menorrhagia are believed to be experienced by a significant segment of the female population. Accordingly, a number of treatments have been developed over the years to remediate this condition. One radical procedure, i.e., hysterectomy, requires the complete surgical removal of the uterus. This surgical procedure has been the treatment of choice in the past and continues to be the ultimate solution if this condition is otherwise non-responsive. Because of the extremity and seriousness of this operation, both in terms of physical and mental effects, attempts have been made to develop less invasive, less radical approaches to relieving menorrhagia.

Less invasive, endometrial ablation treatments have been typically directed at inducing necrosis of the endometrial layer and a portion of the myometrial layer. Known procedures include, inter alia, mechanically scraping the endometrial surface, also known as D&C, freezing of the endometrial layer cryogenically, cauterizing the endometrial layer of the uterus by means of a laser hysteroscope, treating the uterus with microwave generated heat, and ablating the endometrial tissue with an electrosurgical probe. In addition, another known technique involves necrotizing the endometrial tissue by the application of heat, for example, using a liquid filled expandable balloon or directly contacting the endometrium with hot liquid.

The existing cryogenic methods typically require a device having a probe or an extendable bladder which is inserted into the uterus and filled with a circulating gas or fluid at cryogenic temperatures. The cryogenic coolant is typically liquid nitrogen or Freon which is maintained at a sufficient pressure to expand the bladder to be in close contact with the endometrium.

Another technique involves heating the endometrium with microwave energy. This technique has proven to be complex and possibly unreliable because of the irregular shape of the uterus, which makes even energy distribution difficult.

Another known treatment technique utilizes a balloon and heated liquid. The balloon is mounted to the distal end of a catheter that is inserted into the patient's uterus. The balloon is inflated with a liquid, such that the walls of the balloon are substantially in intimate contact with the endometrial layer of the uterus. The liquid is then heated to an elevated temperature so as to cause necrosis and ablation of the cells on the endometrial surface. The liquid may also be heated prior to inflation of the balloon. Fluids such as heated water are utilized as a heating means. U.S. Pat. No. 5,084,044 describes a method for the ablation of tissue in which a distensible balloon, affixed to the end of a catheter, is inserted into a body cavity and inflated using a source of externally heated liquid.

U.S. Pat. No. 4,949,718 discloses an apparatus for effecting necrosis of a tissue lining of a body cavity, specifically the uterine endometrium, by introducing a distensible bladder connected to a catheter into the uterus. The bladder is expanded by introducing a nontoxic, biocompatible fluid under pressure, heating the fluid in the bladder by means located internal to the bladder and controlling the pressure of the fluid and its temperature. U.S. Pat. No. 5,105,808 discloses a method of using this apparatus to effect cauterization necrosis of the uterine endometrium and other body cavities. U.S. Pat. No. 5,460,628 discloses a balloon treatment apparatus with a means for agitating the fluid within the extended balloon in order to better control the heat to which the endometrium is exposed.

U.S. Pat. No. 5,653,692 discloses an endometrial ablation device in which heated fluid contacts the endometrial layer directly. The fluid is introduced at about room temperature and is heated within the uterus by means of RF electrodes.

(C) Breast Tumor/Breast Cancer:

The discovery of a lump in the breast usually brings the thought of cancer immediately to mind. However, it is important to remember that 80% to 85% of all breast lumps are benign, especially in women less than age 40 to 50. Benign causes include fibrocystic breast changes, fibroadenoma, fat necrosis and breast abscess.

Most breast lumps are benign, as in fibroadenoma (Breast mass; Fibrocystic breast disease Abnormal breast mass), a condition that affects mostly women under age 30. Fibrocystic breast disease (FBD) is present in over 60% of all women. The cysts in FBD change in size with the menstrual cycle, whereas a lump from fibroadenoma does not. While most breast lumps are benign it is important to identify those that are not. If a lump is new, persistent, growing, hard, immobile and/or causing skin deformities, or a palpable lump(s) felt in the tissue of one or both breasts it should be evaluated by a health care professional. Such breast lumps may be either benign (non-cancerous) or malignant (cancerous).

The choice of initial treatment for biopsy confirmed breast cancer is based upon the extent and aggressiveness of the disease. Currently, breast cancer is viewed as a systemic disease that requires both local and systemic treatment. Local treatment may include lumpectomy, mastectomy (partial, total, or radical with axillary dissection), and radiation therapy, all directed at the breast and surrounding tissue. Systemic treatment includes chemotherapy and hormonal therapy, which circulate throughout the entire body in an attempt to eliminate cancer cells that may be present in distant parts of the body. Most women receive a combination therapy including surgery, radiation, chemotherapy, and hormonal therapy. Therapy will depend on the extent of the local disease, if there is cancer in local lymph nodes or in other parts of the body as well as the genetic findings after analyzing the cancer cells.

The above discussions described a number of thermal and cryo energy based treatments using a variety of heat sources including RF, Microwave, laser, heated balloons and hot water that are being marketed for tissue necrosis of tumors and fibroids, and for accomplishing endometrial ablation of the uterine cavity. The conventional method of delivery of treatment substances of pharmaceutical drugs is by systemically injecting them into the blood stream with a conventional needle and syringe. This approach severely limits the concentration and formulation of substances that can be injected for treatment of a particular body organ, because the entire body is subjected to the concentrated substance, and therefore the patient must be able to tolerate the dosage. In many cases, it would be advantageous to be able to treat only a particular organ, or a specific part of an organ.

Currently, various laparoscopic/endoscopic surgical and imaging instruments exist that allow a surgeon to view the inside of a body cavity of a patient through a small incision. There are also non-invasive imaging devices including ultrasound, CT, X-Ray and MRI that allow a physician to view body structure details and monitor treatment progress in real time. The use of non-invasive imaging reduces the chances of infection and other complications related to the traditional surgical method employing open and large incisions. The endoscope and non-invasive imaging further allows the surgeon to manipulate microsurgical instruments for controlled tissue ablation of target tissue. Although various microsurgical endoscopic surgical instruments have been developed, the prior art does not describe any apparatus and method for interstitial delivery of treatment substances directly into a body organ for controlled chemo ablation of target tissue under imaging guidance.

In view of the above discussed disease conditions and their treatments, it is apparent that there is a need for further alternative treatment methods, including a method and apparatus that can deliver a treatment substance interstitially into target tissue of a body organ for disease treatment. There is also a need for specific formulations and compositions of treatment substances designed for maximum effectiveness, with dosage ranges specified that are suitable for treatment of a specific body organ and a particular disease.

### SUMMARY

Briefly, the present invention includes a method and apparatus for interstitial treatment of disease by injecting/delivering a treatment substance/chemo-gel, directly into the target tissue of the body organ, and thereby leaving the remaining body organs relatively unaffected. Specific formulations and composition of treatment substance are provided for each of a plurality of body organs and for specific disease conditions. The typical treatment substance/chemo-gel formulations contain two principle components including an active treatment (therapy) substance, and an inactive binding (carrier) substance for thickening the treatment substance. The specific chemo-gel or viscous substance is formulated for recommended dosage level to carry the active treatment (therapy) substance to a particular body organ for optimum treatment of a specific disease. The method also provides a range of treatment substance/chemo-gel dosage and concentration to be injected into each body organ. Apparatus for injecting/delivering the treatment substance/chemo-gel is also provided that can be used with endoscopic instruments using various delivery approaches.

### IN THE DRAWING

FIG. 1 is a chart illustrating the method of the present invention;

FIG. 2 is a list of treatment (therapy) substances;

FIG. 3 is a list of inactive binding (carrier) agents;

FIG. 4 is a list of electrically conductive materials;

FIG. 5A is a chart with chemo-gel formulation/specifications for treatment of breast tumors, fibroids, and endometrial/uterine cavity ablation with ethanol gel/ethyl alcohol gel;

FIG. 5B is a chart with chemo-gel formulation/specifications for treatment of breast tumors, fibroids and endometrial/uterine cavity ablation with saline gel/hypertonic saline gel;

FIG. 6A is a chart specifying treatment substance formulations and dosages for treating various specific diseases and body organs;

FIG. 6B is a chart specifying treatment substance formulations and dosages for treating various diseases and body organs;

FIG. 7A is a chart of injection treatment parameters illustrating various elements of the injection methods, including the injection delivery methods, imaging guidance methods, and injection substance forms of the present invention for a plurality of body organs and related diseases;

FIG. 7B is a chart of injection treatment parameters illustrating various elements of the injection methods, including the injection delivery methods, imaging guidance methods, and injection substance forms of the present invention for each of a plurality of body organs and related diseases;

FIG. 8 shows injection into each of a plurality of body organs;

FIG. 9A illustrates injection into target tissue of a breast;

FIG. 9B illustrates use of a biopsy needle guide for insertion of a needle delivery apparatus for injecting a treatment substance;

FIG. 10 shows injections into a body organ using a syringe and needle, and use of an endoscope and gel injection apparatus;

FIG. 11A illustrates a transcervical/transvaginal delivery device for injection of a treatment substance, wherein the device can be inserted through a hysteroscope;

FIG. 11B shows further detail of the device of FIG. 11A;

FIG. 12 illustrates an injection needle device inserted into a working channel of an endoscope, which can be used with either a rigid or flexible hysteroscope or resectoscope;

FIG. 13 illustrates the use of an injection/delivery apparatus via flexible hysteroscope, guided through a cervix/vagina by ultrasound imaging, for injecting a treatment substance/chemo-gel into fibroids;

FIG. 14A illustrates accessing a target tissue such as a breast tumor percutaneously or endoscopically, guided by an imaging device for injection of a treatment substance using a syringe;

FIG. 14B illustrates accessing a target tissue such as a uterine fibroid percutaneously, or laparoscopically guided by an abdominal, transvaginal or laparoscopic ultrasonic imaging probe for injection of a treatment substance with a syringe, under non-invasive imaging guidance;

FIG. 15A illustrates accessing a fibroid with a biopsy probe;

FIG. 15B illustrates accessing a fibroid transvaginally or transcervically with an injection/delivery device guided by a biopsy guide probe or transvaginal probe under transvaginal or abdominal ultrasound imaging guidance for injection of a treatment substance, and alternatively with the optional application of RF energy;

FIG. 16 illustrates accessing a fibroid by a hysteroscopic approach for injection of a treatment substance with a syringe under endoscopic imaging guidance;

FIG. 17 shows a transvaginal/transrectal ultrasound probe with a working channel for guiding an injection/delivery needle device;

FIG. 18 shows a transvaginal/transrectal ultrasound probe with an external biopsy needle guide;

FIG. 19A illustrates endometrial ablation treatment by injecting a viscous treatment substance percutaneously, or using a intrauterine delivery catheter, guided by an imaging method and injection apparatus;

FIG. 19B shows a plurality of catheter lumens, and an articulated catheter tip;

FIG. 19C shows a plurality of holes and configurations in a delivery catheter tip;

FIG. 20 illustrates use of a viewing endoscope through a delivery catheter;

FIG. 21 illustrates use of a tissue recognization system with CCD and reflected light to view and analyze uterine cavity and endometrial tissue; and

FIG. 22 illustrates a delivery catheter with a suction cup apparatus for providing a vacuum to seal a plug in the cervix.

### DETAILED DESCRIPTION OF THE PREFERRED EMBODIMENT

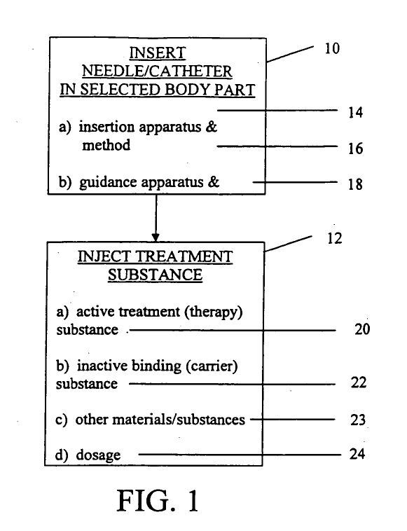

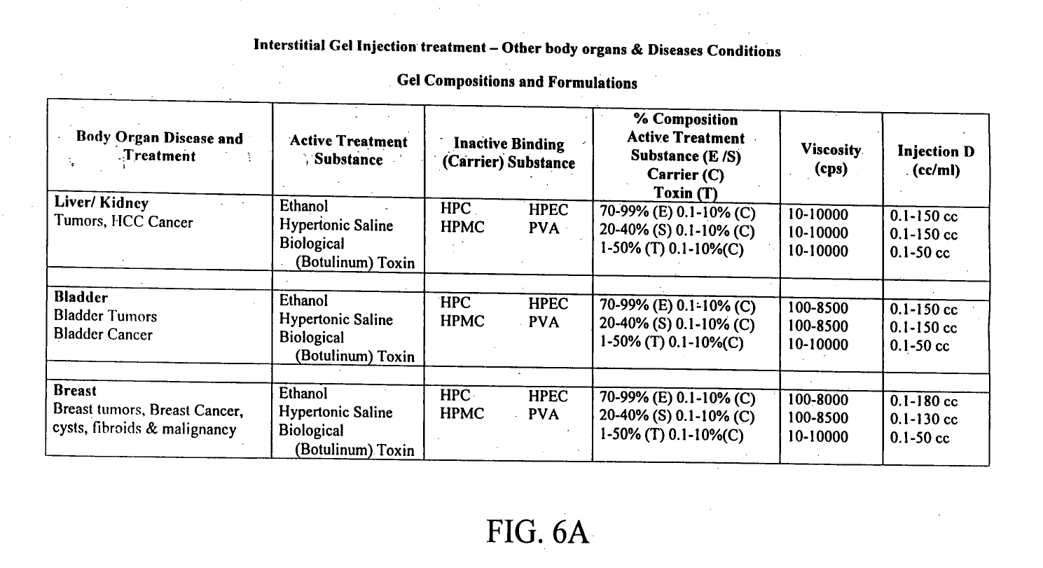

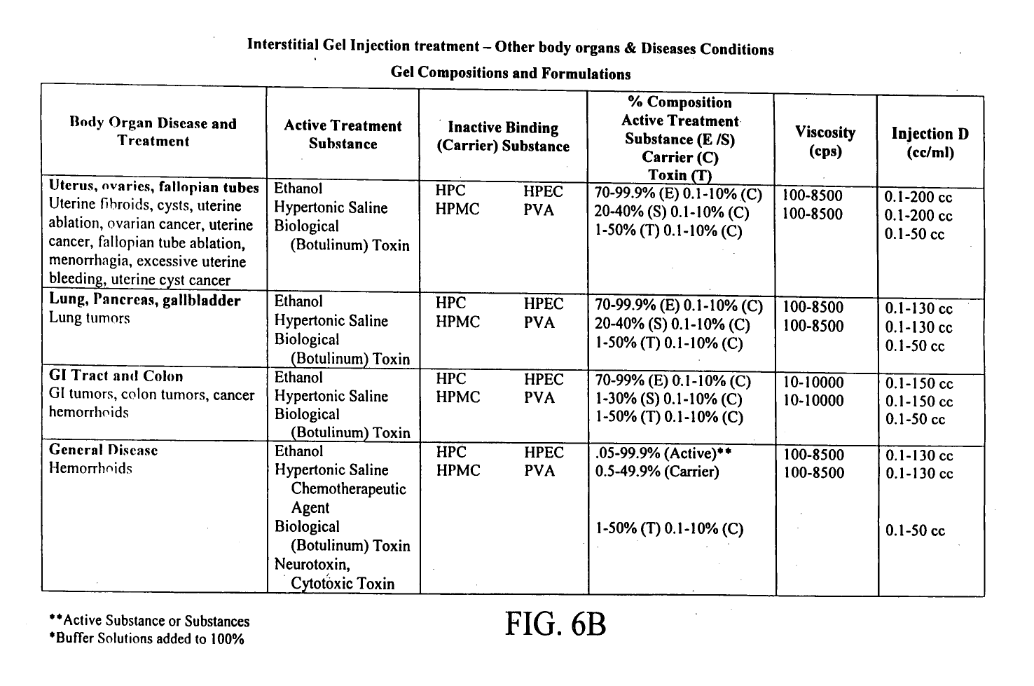

A preferred embodiment of the present invention will now be described in reference to the chart of FIG. 1 of the drawing. According to the method, a hollow core needle or delivery catheter is inserted, by any of various methods and apparatus, into a person's body organ to be treated (block 10). A treatment substance/chemo-gel is then injected through the needle/catheter and into the body organ (block 12), providing a localized application of the substance, leaving the remainder of the person's body relatively unaffected by the substance. The method applies generally to any disease treatable with an injectable treatment substance, including but not limited to a chemo ablation substance and applies to any body organ, including but not limited to the breast, uterus, fallopian tube, ovary, lung, liver, kidney, fibroid, myoma, rectum, bladder, gallbladder, adrenal gland and other body organs for example as listed in FIGS. 6A and 6B.

The apparatus 14 and corresponding methods for insertion of the needle/catheter include any of a variety of surgical instruments and their use, including a rigid or flexible endoscope, falloposcope, hysteroscope, laparoscope and ultrasound probe. The insertion of the needle/catheter is guided by any of a variety of methods and apparatus 16, including but not limited to the scopes listed above, and including other invasive guidance apparatus, and non-invasive imaging methods and apparatus. The methods and apparatus can be, for example, an ultrasound probe, a biopsy guide, a needle guide, template, grid or other positioning and guiding apparatus. Non-invasive methods and apparatus for guiding the needle include ultrasound apparatus, CT, MRI, and X-Ray and Gamma-Ray apparatus.

The present invention includes bringing a needle/catheter to a selected body organ by way of any selected body passage 18, such as through a cervix, vagina or rectum. The needle can also be brought to the selected body part through the skin, or through an incision in the skin and other means. For example, a needle/catheter can be brought into the uterine cavity percutaneously through the skin of the abdominal area or vaginal area, and then interstitially into the uterus.

The injected treatment substance/chemo-gel includes an active treatment (therapy) substance 20 and an inactive binding (carrier) substance 22 that carries the active treatment substance for controlling the dispersion of the treatment substance once injected into the body organ. The active treatment substance 20 can be any material substance included for a particular active/treatment tissue effect. In addition to the carrier and active material, the substance can include other material 23 to provide required physical properties of the treatment substance for any non-active purpose. The treatment substance, for example, can include elements in any form, such as liquid, gas, solid, gel, viscous fluid, semi-liquid solutions, semi-solid, suspensions, colloids, foam, paste, beads, pallets, micro spheres and conjugates. For the purpose of generalization, the term “inactive binding substance” or “carrier” will be used to refer to both viscous and gel material. The inactive binding substance 22 slows the rate of dispersion, reducing the overall volume of tissue treated by the active treatment substance and thereby increasing the concentration of the treatment substance in the target zone of the body organ for a given dosage 24. The concentration of the active substance in a body organ depends in part on the viscosity factor of the inactive binding substance as discussed above, wherein a more viscous substance will disperse more slowly and therefore result in a higher concentration in the target zone of the body organ. The concentration of the active treatment substance also depends on the percentage of the active treatment substance in the treatment substance i.e., the ratio of the active substance to the inactive substance. These parameters will be discussed more completely in the text in reference to the following figures of the drawing.

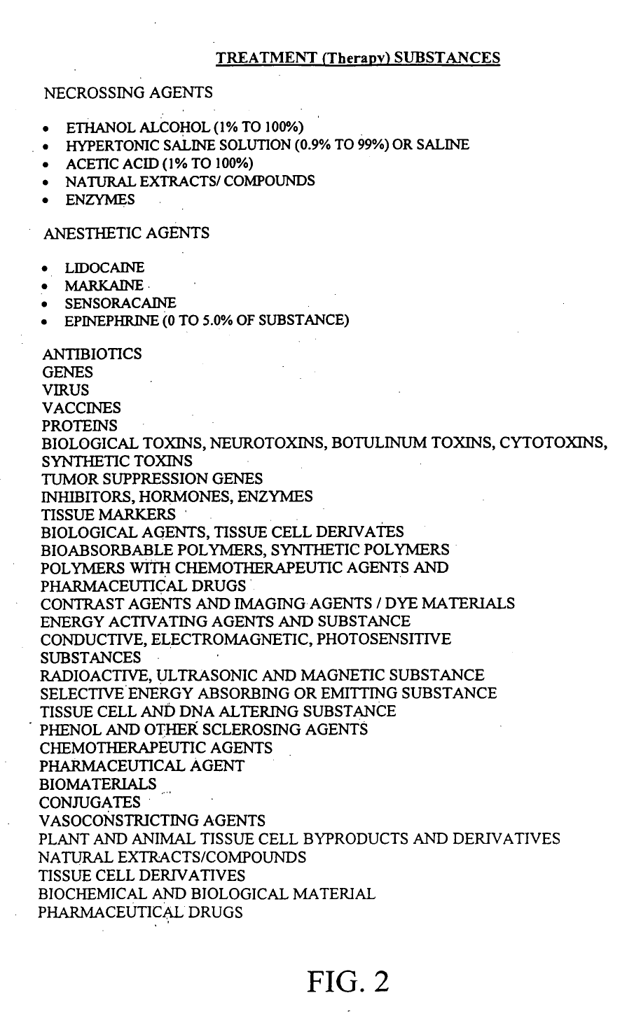

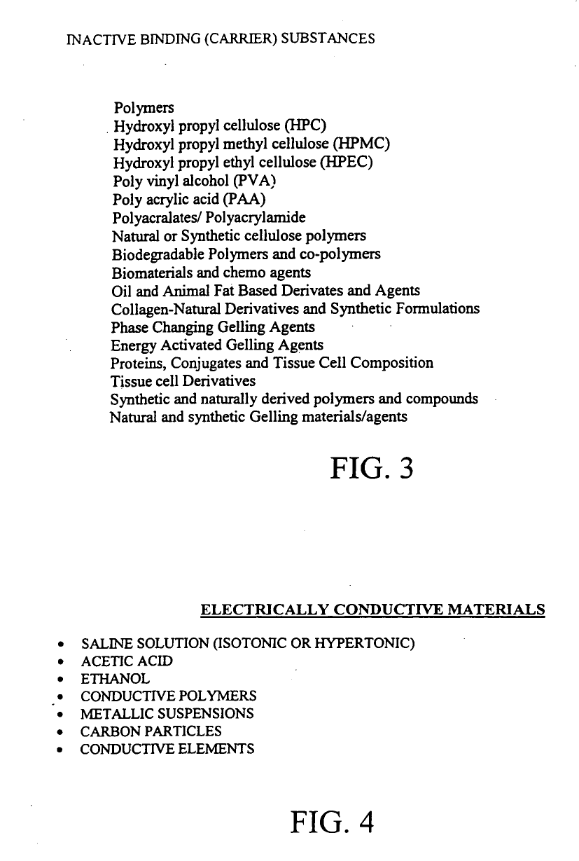

FIG. 2 is a list of active treatment substances, and FIG. 3 is a list of inactive binding (carrier) substances. One or more of the active treatment substances 20 of FIG. 2 and one or more of the inactive binding substances 22 of FIG. 3 may be selected and combined to form a treatment substance for interstitial injection treatment. FIG. 4 is a list of electrically conductive substances that can also be added to the treatment substance or applied separately in the event that application of RF (radio frequency) energy is desired for treatment.

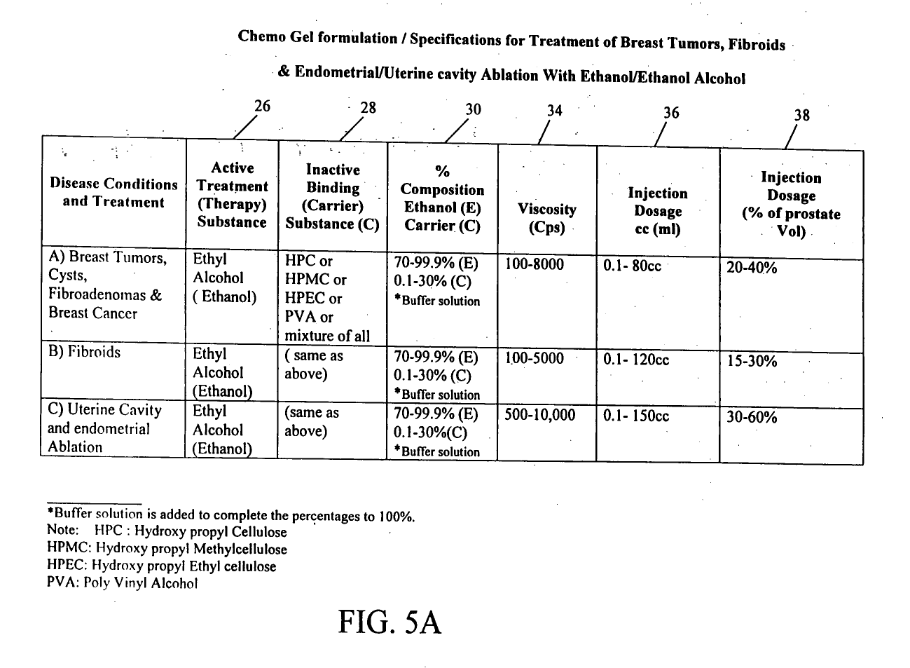

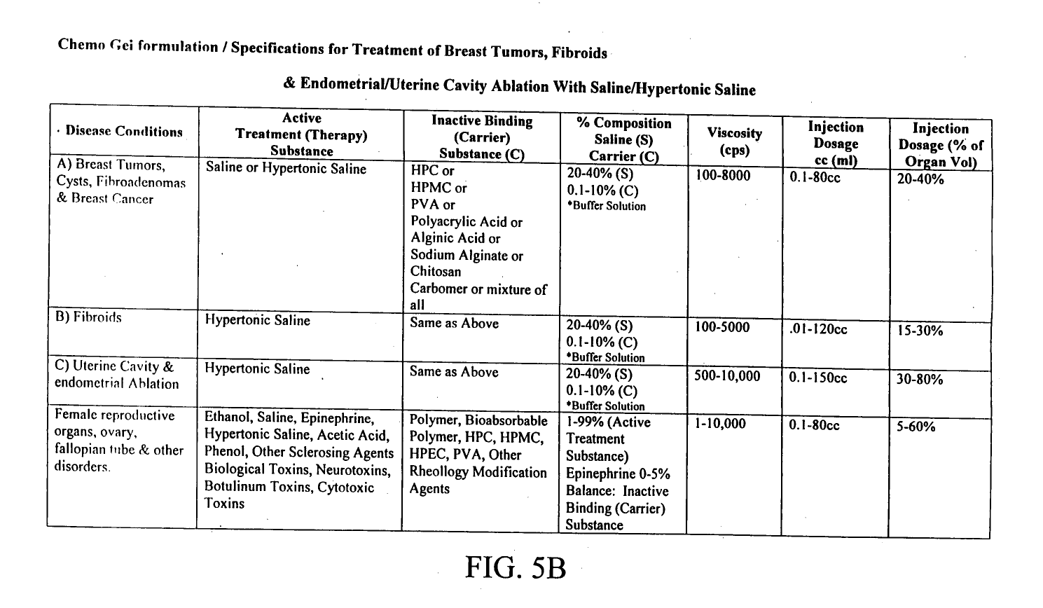

Specific treatment substance formulations for treating diseases of the breast, fibroid and uterine cavity are detailed in FIGS. 5A and 5B. FIG. 5A includes formulations using ethanol/ethyl alcohol as the active substance (column 26) for treatment of breast tumors, cysts, fibroadenomas, fibroids, uterine cavity, and for endometrial ablation.

The inactive binding substance/carrier (gelling/viscous agent) is selected from the group listed in column 28, and includes the polymers HPC, HPMC, HPEC and PVA in any combination. The treatment substance includes the combination of at least one inactive substance and at least one active substance. The resultant treatment substance is indicated in column 30.

It should be noted in reference to FIGS. 5A-5B, and 6A and 6B that the percentages in the composition columns do not in all cases account for 100% of the treatment substance. In these cases, the remaining percentage is to be assumed to include a buffer solution. For example, in the first row of FIG. 5A for treatment of fibroids, column 30 shows a possible 70% ethanol, and a maximum carrier (C) substance of 20%. Since 70% plus 20% is only 90%, the balance of 10% can be a buffer solution or a neutral substance, either active or inactive as an alternate embodiment. This logic applies to all of the composition data in the various tables of the present specification. It should also be noted that although FIGS. 5A-5B and 6A and 6B show specific ranges for specific active and inactive substances, other combinations are also included in the spirit of the present invention. For example, a single treatment substance may include more than one active substance, such as a combination of ethanol and a saline solution, or ethanol and epinephrine.

Referring specifically to FIG. 5A, as an example a treatment substance for injection treatment of fibroids includes an active substance of 70-99.9% ethanol, and 0.1-20% inactive binding (carrier) substance. The viscosity (column 34) for fibroids is preferred to be in the range of 100-5000 cps. The injection dosage (column 36) for fibroids is 0.1-120 cc, and in column 38 the injection dosage as a percentage of fibroid volume is in the range of 15-30%. FIG. 5B is a chart of formulations for the disorders of breast, fibroids and uterine cavity as in FIG. 5A, except the active substance is saline or hypertonic saline. For treatment of general female reproductive organ disorders, the treatment substances include at least one active treatment substance selected from the list in an amount equal to 70 to 99% of the treatment substance. The inactive binding materials are listed. Epinephrine is included in the formulation from 0 to 5% of the treatment substance. The preferred viscosity is 1-10,000 cps, the dosage 0.1-80 cc, and the volume of prostate treated is in the range of 20-60%.

FIGS. 6A and 6B list treatment substance formulations for use in treating specific diseases of body parts including a liver, kidney, bladder, breast, uterus, ovary, fallopian tube, lung, pancreas, gallbladder, G.I. tract and colon. In addition to the preferred specifications listed, FIG. 6B provides a formulation for treatment of any disease including 0.05-99.9% of an active substance such as ethanol, saline, or chemotherapeutic agent, biological toxins, neurotoxins and 0.05-49.9% carrier inactive binding substance. The inactive binding (carrier) substance in FIG. 6B can be a polymer (P), or other material/substance to provide the required binding (gelling/viscous) carrier property desired. The general disease category of FIG. 6B includes treatment of diseases including vocal cord disease, pancreatic cancer, myomas, gastric tissue growth, gastric cancer and tissue growth, and any unspecified tumors and disorders, including the diseases of other organs listed in FIGS. 6A and 6B. The active treatment substance, chemotherapeutic agent and biological toxin, for example, can be for the purpose of tissue or nerve destruction.

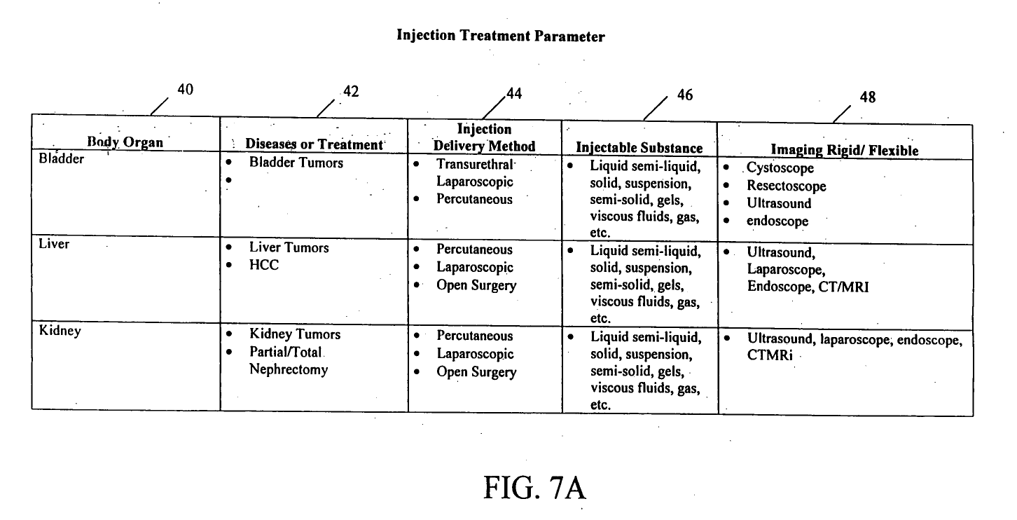

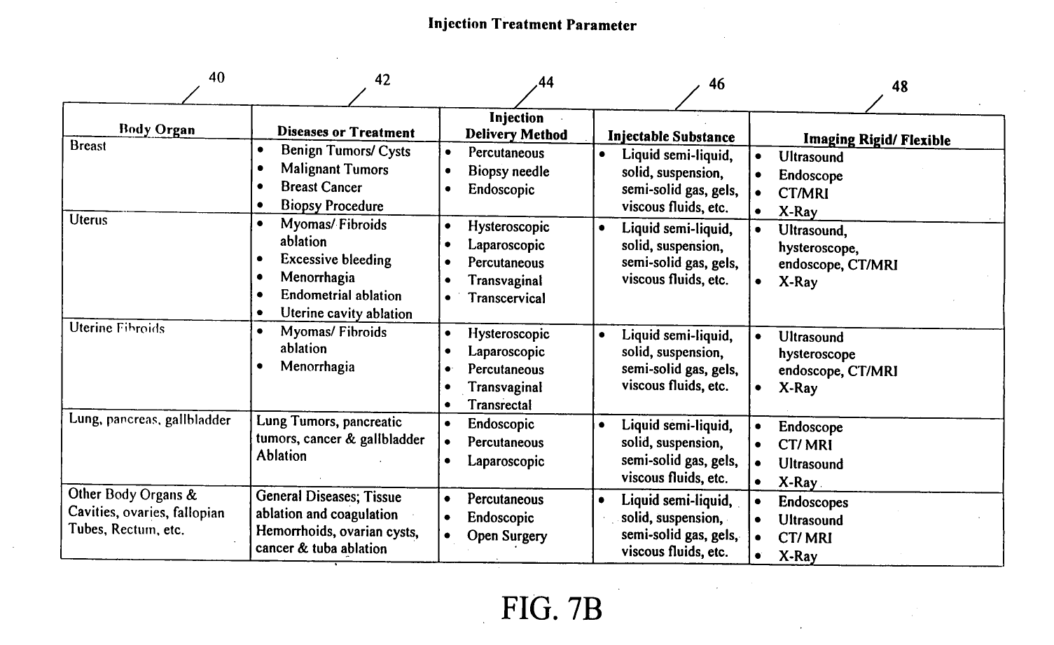

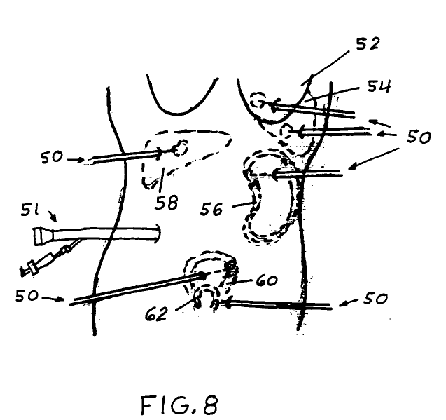

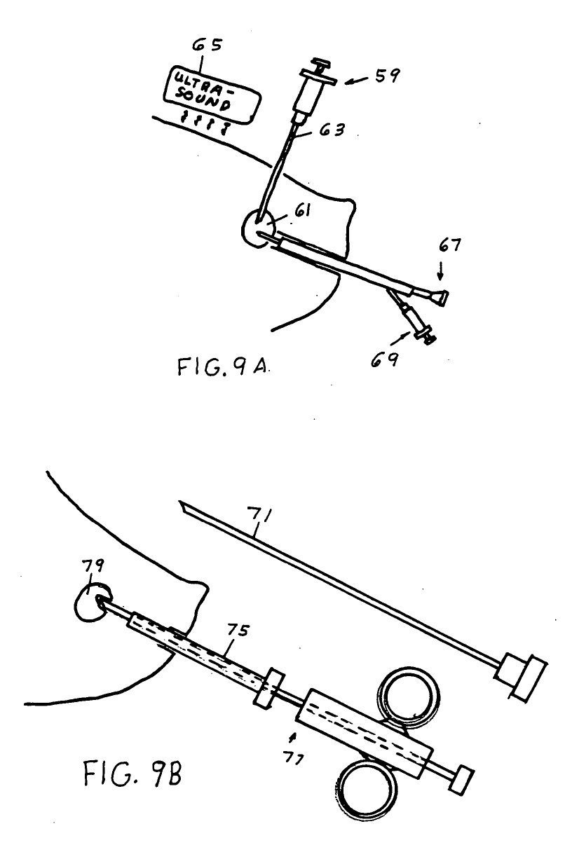

FIGS. 7A and 7B are a chart summarizing the method and apparatus for injection treatment of body parts including bladder, liver, kidney, breast, uterus, uterine fibroids, lung, pancreas, gallbladder, and other body organs. Column 40 indicates the particular body organ. Column 42 includes the diseases and/or treatment for each organ. Column 44 summarizes the passages through which apparatus including the hollow core injection needle are conveyed for delivering the needle to the particular organ. Column 46 lists various forms of the elements that can be part of the treatment substance. Although FIG. 1 indicates that the embodiment of the treatment substance includes an inactive substance/carrier that is a gelling/viscous agent in combination with an active substance, the form of the injected treatment substance can alternatively be any combination of the forms indicated in FIGS. 7A and 7B, including for example a solid and a gas. Column 48 lists methods and apparatus that can be used in the process of guiding the needle/catheter to the selected body part. These lists include invasive and non-invasive guiding apparatus.

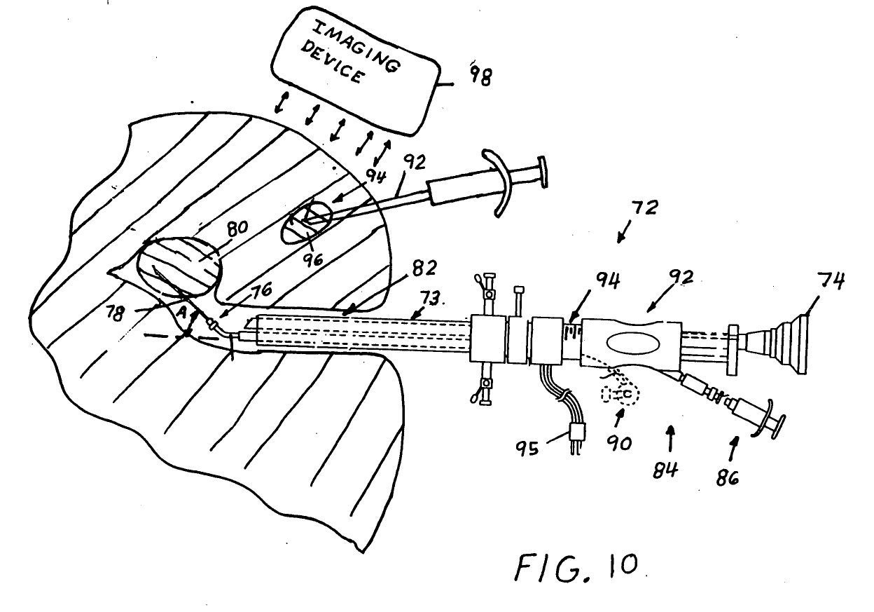

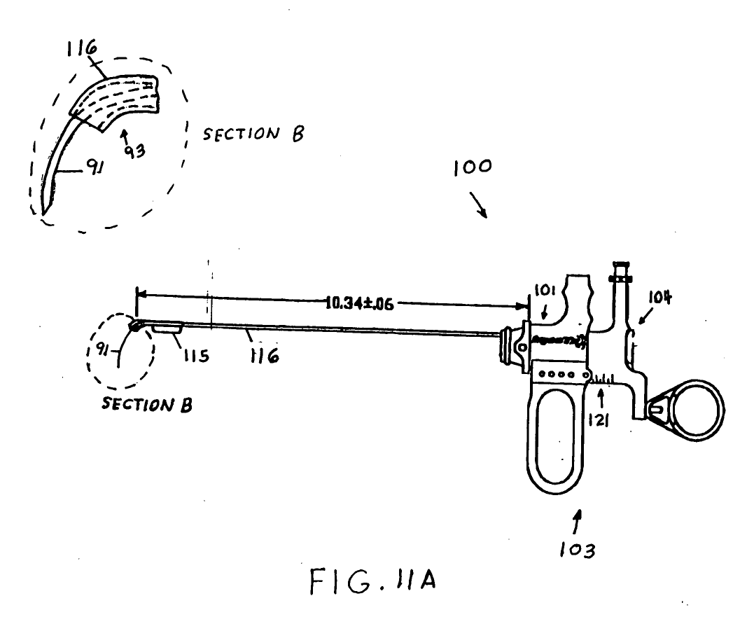

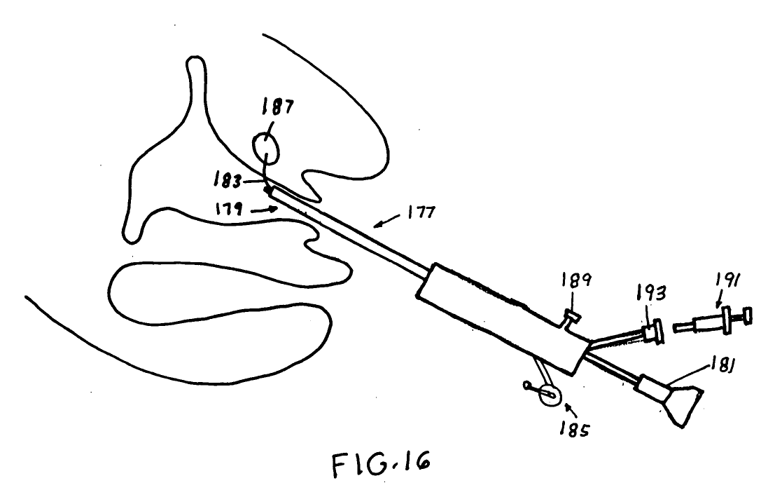

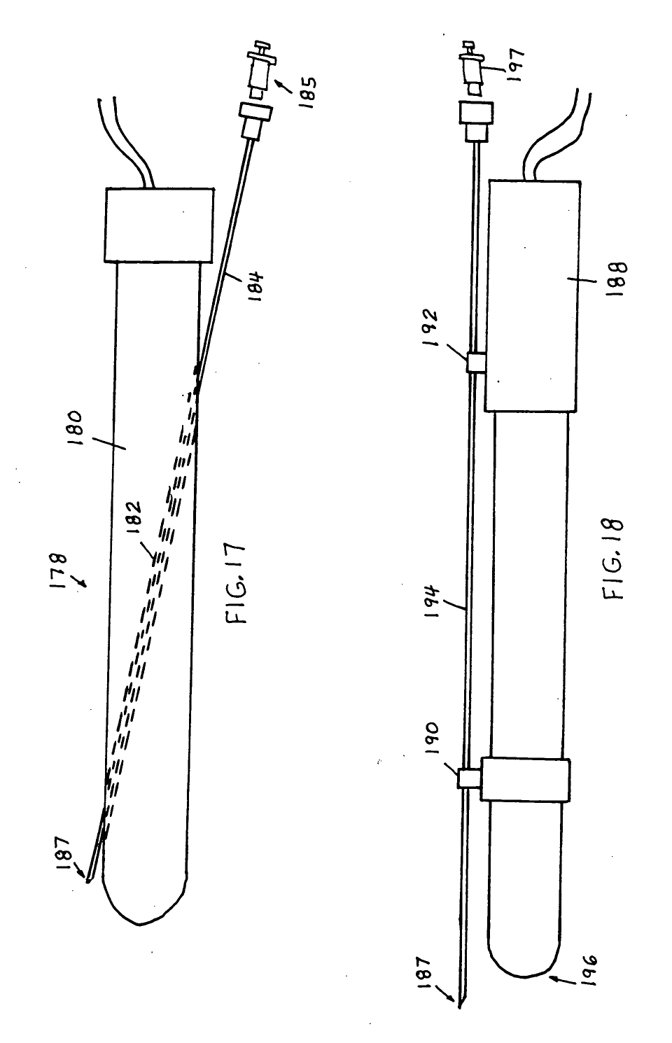

Application of the present invention is illustrated for various body organs in FIG. 8, showing percutaneous access to organs of a body for injecting/delivering a treatment substance. Injection devices 50 are shown figuratively in FIG. 8 for treatment of a breast 52, lung 54, kidney 56, liver 58, uterus 60, and bladder 62. The devices 50 can be of various configurations for access to a target tissue in need of treatment. An injection needle is included in the device 50 for injection of a treatment substance according to the present invention. The apparatus 50 can also include any of the various devices described in the present disclosure or referred to in this disclosure, such as an endoscope generally described in FIG. 10, with more particular devices described elsewhere, such as in FIGS. 11A, 11B, 12, 13, 14A, 14B, 15, 16, 17 and 18. In addition, a biopsy probe including a delivery channel can also be used to insert the hollow core needle of the present invention. Details of a biopsy probe will be understood by those skilled in the art, and need not be described herein. Such a device can be used at any point of entry, including a natural opening or an incision. As another example, the endoscopic intracavity ultrasound probes of FIGS. 17 and 18 can be dimensioned for use in accessing any of the various body parts. In further example, if the endoscope instrument 51 is replaced with one of the devices of FIGS. 17 and 18, the device could be appropriately described as an abdominal ultrasonic probe with a needle guide. The needle tip in all of the devices disclosed herein can alternatively be echogenic so as to be easily visible with an imaging method. Also, the various methods of guidance apply to FIG. 8, such as use of an endoscopic instrument and/or an imaging method. The endoscopic instrument 51 of FIG. 8 is similar to endoscope 79 of FIG. 10. The endoscope 51, placed for access to the abdominal peritoneal cavity, can also be referred to as a laparoscope, and because the needle inserted percutaneously enters the target tissue, the device 51 is generally referred to as a percutaneous or laparoscopic device.

The treatment of fibroadenomas, including benign breast tumors and cysts according to the present invention is illustrated in FIG. 9A, and involves chemo ablation of the lump 61. The chemo ablation procedure is preceded by screening against cancer of the breast lump. A tissue biopsy of the suspicious tumor must exclude cancer or precancerous lesions of the breast. The size and location of the breast lump can be determined by use of a non-invasive imaging device such as ultrasound, etc. An injection needle/catheter delivery device 59 of the present invention can be inserted percutaneously to the lump 61 to administer the viscous treatment substance for chemo ablation. The needle 63 can be guided in various ways, including use of non-invasive imaging apparatus such as an ultrasound imaging device positioned adjacent the breast, symbolized by block 65 labeled ultrasound imaging, but can also be another type of imaging device such as CT, MRI, X-Ray, etc. An endoscope 67 apparatus can be used, and inserted through an incision along with a needle and syringe apparatus 69 as illustrated in FIG. 10 for any organ. As shown in FIG. 9B, an injection needle 71 can also be inserted through an introducer sleeve 75 of a breast biopsy device 77 into target tissue 79, following a biopsy procedure. This method avoids the need for a physician to make an additional puncture for injecting the treatment substance. Subsequent to initial insertion of the injection needle device in the breast tumor, the position of the needle and its depth in the target tissue can be confirmed by real time ultrasound imaging. The physician can also monitor the actual volume of the chemo-gel injection into the breast tissue in “real time” using an ultrasound imaging probe/device. The chemo-gel can cause controlled tissue ablation of the breast tumor under imaging guidance without creating any adverse effect on surrounding organs. The chemo-gel treatment substance concentration, composition and formulation and recommended dosage based on tumor volume are outlined in FIG. 6A.

FIG. 10 shows the use of an endoscopic instrument 72 equipped with a scope 74 separately inserted through the instrument 72 housing 73 for viewing inside a body cavity 76 for visual guidance in directing a needle 78 into a target tissue 80. FIG. 10 as shown is meant to illustrate the general use of an endoscope on any body part. The endoscopic instrument 72 is shown inserted into a canal 82 which is representative in FIG. 10 of any body opening, natural or fabricated. The endoscope 72 therefore represents any of the variety of endoscopes, such as a hysteroscope, laparoscope and resectoscope. Because the needle enters the target tissue interstitially, the various endoscopes (hysteroscope, laparoscope and resectoscope, etc.) can also be described as an interstitial endoscopic device. The delivery channel for guiding the needle through the endoscope can be referred to as a working channel. The instrument 72 as shown includes a treatment substance injection apparatus 84, including the needle 78, and an injector 86, represented as a syringe type of device. The Instrument 72 includes a sliding mechanism 92 to move the needle 78 forward into the tissue 80. The needle depth is controlled by visual marking 94 on the sliding mechanism handle. Furthermore the instrument 72 has an RF connector (optional) attachment 95 for application of RF energy. Further details relevant to the instrument 72, for example a device 90 for controlling the needle 78, are included in U.S. patent Ser. Nos. 09/510,537 and 09/715,853, the contents of which are incorporated in the present disclosure by reference. FIG. 10 also shows a needle 92 percutaneously inserted and through interstitial tissue 94 to a target material 96. FIG. 10 symbolically illustrates guidance of the needle 92 to the target tissue 96 with a non-invasive imaging guidance device indicated by block 98. Those skilled in the art will know how to incorporate such apparatus 98 for the purpose of guiding the needle 92. The imaging device can be ultrasound, X-Ray, MRI, CT, etc.

FIG. 11A is a scaled drawing that shows a hysteroscopic, transcervical/transvaginal injection device 100, with retractable curved needle 91 designed for injection of a viscous treatment substance with a commercially available model of rigid hysteroscope 100. A length 93 of the needle guide tube 116 can be curved as shown in more detail in the enlarged section B view, for aiding the extension of the curved needle 91 from the tube 116.

FIG. 11B is an enlarged and simplified cross sectional view of the device 100 of FIG. 11A with the addition of a scope probe 117 installed. FIG. 11B is purposely not drawn to scale so that the various parts can be more clearly illustrated. The apparatus 100 is designed with a needle advancing mechanism included in first and second apparatus as follows. The first apparatus 101 has a channel 102 dimensioned for a sliding fit with the body 103 of the second apparatus 104. The second apparatus 104 has the hollow core needle 91 attached, with a proximal end 105 installed in fluid connection with a treatment substance channel 106 that can be fed by a treatment substance supply 120 connected to the channel 106 through a connector 107. The needle 91 is optimized for controlled delivery of a treatment substance including an inactive binding (carrier) substance as well as an active (therapy) substance as set forth in the various text and figures of the present specification. FIG. 11B also shows an electrical connection 108 in contact with the needle 91 for application of RF energy as an alternate embodiment, not shown in FIG. 11A. The second apparatus 104 also includes a channel 109 for passage of a cystoscope 110 as a separate device that can be used with the apparatus 100, and shown in FIG. 11B for illustration. The second apparatus 104 includes a thumb ring 111 or other device for allowing an operator to move the second apparatus 104 relative to the first apparatus 101 by simultaneously gripping the ring 111 and slotted handle 112, allowing an operator to move the first apparatus relative to the second apparatus as indicated by the two way arrow 113. The first apparatus includes a needle guide channel 114 for passage of the needle 91, and a scope clip 115 for positioning the scope 110 probe 117 relative to the first apparatus 101. The end proximate 118 of the tube 116 and needle 91 shown in FIG. 11B are both straight. Alternatively, the tube 116 can have an end portion 93 as shown in FIG. 11A, and in the dashed lines of FIG. 11B, that is curved in any desired direction for aiding in extending a pre-stressed, curved needle 91 as indicated by the dashed lines, for use in puncturing tissue that is off to a side of the tube 116 axis.

In operation, the tube 116 with needle 91 and optionally the scope probe 117 are inserted into a body passage such as a cervix/vagina. When the end 118 of the tube 116 is in the desired position near uterine fibroid tissue to be treated, an operator moves the needle 105/91 forward by pushing the ring 111 towards the handle 112, forcing the body 103 of the second apparatus 104 into the channel 102 of the first apparatus, driving the straight or curved needle tip 119/129 into the desired tissue. The operator then activates a treatment substance injection device 120, such as a syringe attached to the connector 107, to push the treatment substance through and out of the needle 105 into the target tissue. The position of the tube end 118 and needle tip 119 can be observed either through use of the endoscope 110 or through use of a non-invasive imaging device such as illustrated in FIG. 10, or a combination of the two methods. The depth of penetration of the needle into the fibroid can be monitored through use of a non-invasive imaging device and/or through use of calibration/depth marks on the apparatus 100, such as at 121 (FIG. 11A), indicating the relative positions of the first and second apparatus 101 and 104. To enhance non-invasive imaging of the needle position, the area of the needle tip 119 is alternatively constructed to include echogenic properties, which is also discussed elsewhere in the present disclosure.

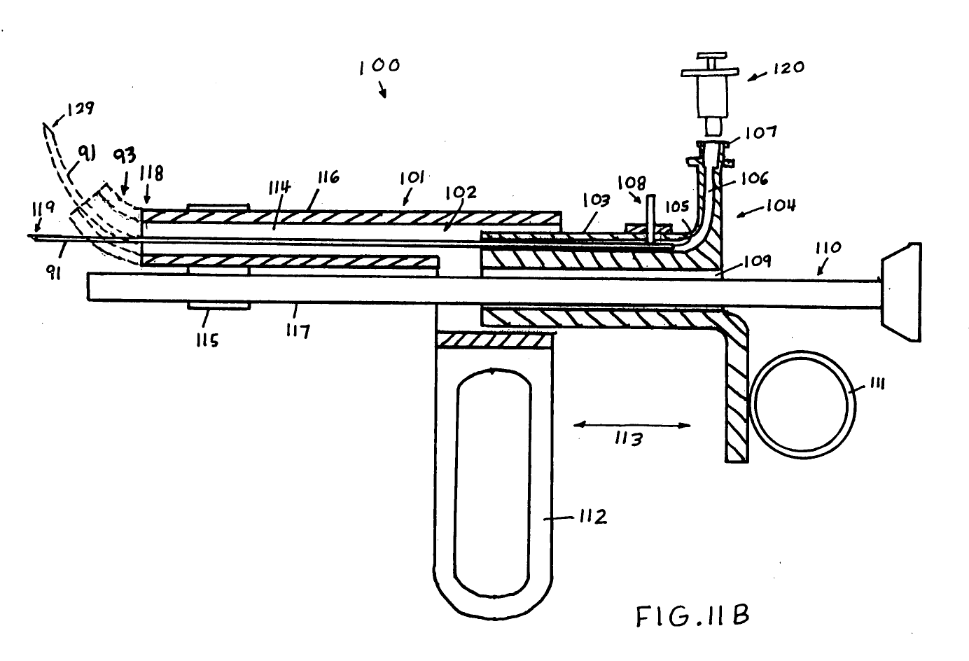

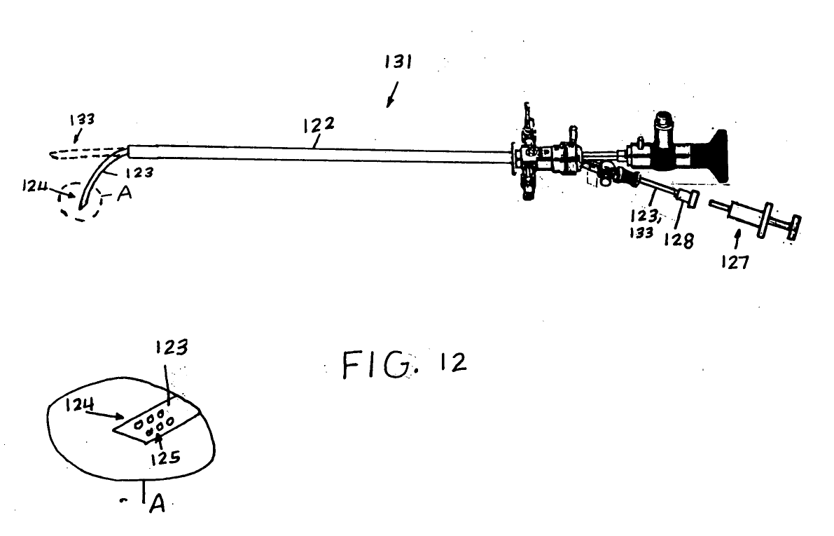

FIG. 12 is a view of an endoscopic apparatus 131, similar to the apparatus 72 of FIG. 10, except the relative dimensions are correctly shown for an actual working hysteroscopic apparatus, but not drawn for ease of illustration of the various parts. For a detailed description of the working apparatus, refer to FIG. 10 and the corresponding description. FIG. 12 shows the hysteroscopic apparatus 131 as having a long, slender tube 122, which can be either rigid or flexible. The apparatus 131 includes an injection needle which can be curved at 123, or straight as in dashed lines 133, and configured (length and diameter) for optimum injection of a treatment substance having an inactive binding (carrier) substance and an active treatment substance as described in the various figures of the present disclosure. The tube 122 can be inserted, for example through the vagina and cervical canal into the uterus, and the injection needle 123/133 can then be deployed into a uterine fibroid manually under endoscopic visualization for injection treatment, and/or can be guided by an imaging method as described in reference to FIG. 10 above. If imaging is used, the injection needle tip 124 is designed for high echogenecity, and as shown in the expanded Section A of FIG. 12, with one or more holes 125 with various sizes and patterns for optimum distribution of the treatment substance for a desired tissue effect. The injection needle can also be made from super elastic materials for curved or angular tip articulation. FIG. 12 shows a treatment substance injection device 127, illustrated symbolically as a syringe for connection to the needle by way of connector 128.

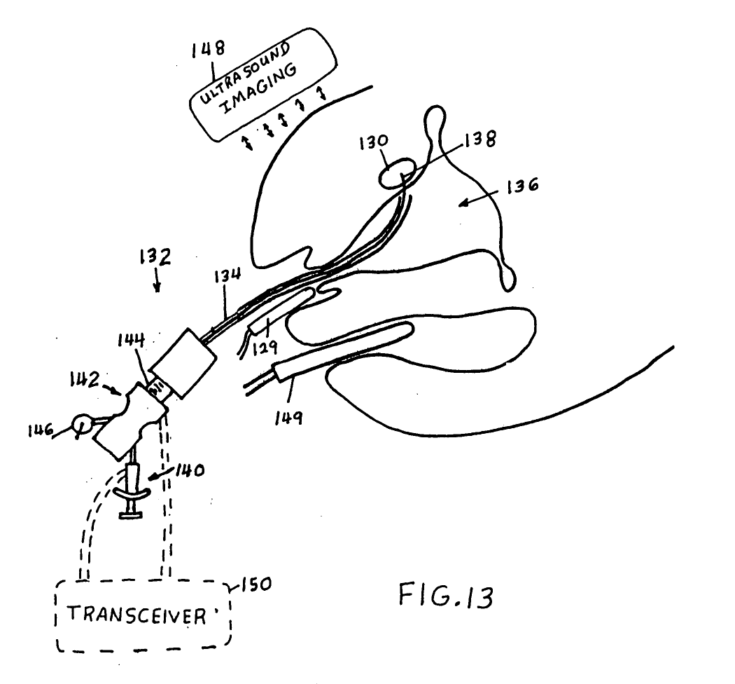

Transvaginal, hysteroscopic access to a uterine fibroid 130 is illustrated in FIG. 13. A hysteroscopic instrument 132 probe 134, which can be either rigid or flexible, is inserted into the uterine cavity 136 via cervix. One or more hollow core needles 138 are inserted through a working channel of the probe 134. The gynecological instrument can be, for example, a rigid or flexible endoscope such as a hysteroscope, resectoscope, etc., and can be a special/novel design, or any of a variety of commercially available instrumentation. The instrument 132 has a substance injection device 140. The depth of the needle 138 is controllable by an adjustment device 142 and scale 144. A needle curvature adjustment apparatus is symbolically represented by item 146. Further details of these features of the device 132, including ultrasound imaging device 148 and/or transvaginal/transrectal imaging probe 149 and transceiver 150 are described in U.S. patent application Ser. No. 09/510,537 incorporated by reference. The transceiver 150 can be for operation of a transluminal ultrasound probe inserted into the vagina and uterus cavity through a channel in the probe 134. The ultrasound imaging device 148 is shown positioned adjacent to the abdominal area, and is more correctly described as an abdominal ultrasound imaging device 148. As shown in FIG. 13, an ultrasound imaging probe may be placed in any of various positions, including the abdominal ultrasound device positioned adjacent to the abdomen, a transluminal ultrasound probe inserted through the hysteroscopic probe 134, a transvaginal probe 129 in the vagina, and a transrectal ultrasound probe 149 in the rectum.

A percutaneous device 152 is shown in FIG. 14A for percutaneous access to a breast tumor 151, and to a uterine fibroid 165 in FIG. 14B. The percutaneous device is designed to be used independently or in conjunction with guide templates, grids, guides 156, or other positioning/guiding apparatus including imaging devices such as ultrasound, X-ray, etc. In some cases, it is possible to visually see a lump, or to palpate/feel it sufficiently to guide the needle. FIGS. 14A and 14B are convenient to illustrate that needle passage can proceed through various tissue types. The needle 158 of FIGS. 14A and 14B can pass through skin 153, 169, vesicles 155, 157 and interstitial space 159 and 161. Alternatively, the percutaneous injection needle 158 device, preferably 22-14 gauge size has an echogenic tip 160, an image enhancement feature that is desirable for injection treatment as applied to all other body organs, and is designed to aid the injection of a treatment substance in the form of a gel and/or a viscous substance as discussed in the various figures and text of the present disclosure into the tumor 151, fibroid 165 under ultrasound imaging guidance. The ultrasonic guidance device includes an ultrasonic imaging probe placed in near proximity to the organ to be injected. This can be done, for example by placing an ultrasonic imaging probe 162 in the rectum 164, or an imaging probe 171 in the vagina 173, or an imaging probe 175 adjacent to the abdomen 300. The injection device needle tip 160 can be straight, curved, angular or articulating to direct the injection, such as in the uterine cavity anatomy. The injection needle device 152 and positioning/guiding template 156 can be designed to allow semi-automatic or automatic injection treatment operation and a programmed dosage plan using computer software and an automatic needle advancement and retraction mechanism. The treatment substance of the present invention can alternatively include in addition to the materials described above, an agent or an element providing a hyper echoic characteristic, making it visible under ultrasound, CT or MRI imaging. The actual location of injectable treatment substance in the target tissue and extent of volumetric coverage in situ can be monitored on a “real time” basis using ultrasound or other imaging device, as symbolically represented in FIG. 14A by block 167, and imaging probe 162 in FIG. 14B. The injection needle tip 160 and treatment substance are visible as a bright white echogenic reflection, which can be controlled by adjusting the injection dosage volume in an interactive mode. The target tissue can be destroyed by injecting a treatment substance including an active substance, and an inactive binding substance; i.e., a thickened carrier (gel or viscous) substance under ultrasound imaging guidance. The typical injection dosage of treatment substance for fibroids varies between 10-30% of its volume measured by ultrasound imaging. The use of a treatment substance with a hyper echoic property and/or a hyper echoic needle tip, visible under non-invasive imaging applies as an element in an alternate embodiment for injection of any organ or method as described in the present disclosure. FIGS. 14A and 14B also are used to illustrate access to a body organ using a laparoscope through an incision, simply by replacing the percutaneous device 152 as illustrated with an endoscopic laparoscope or laparoscopic ultrasound probe, to be inserted through an incision.

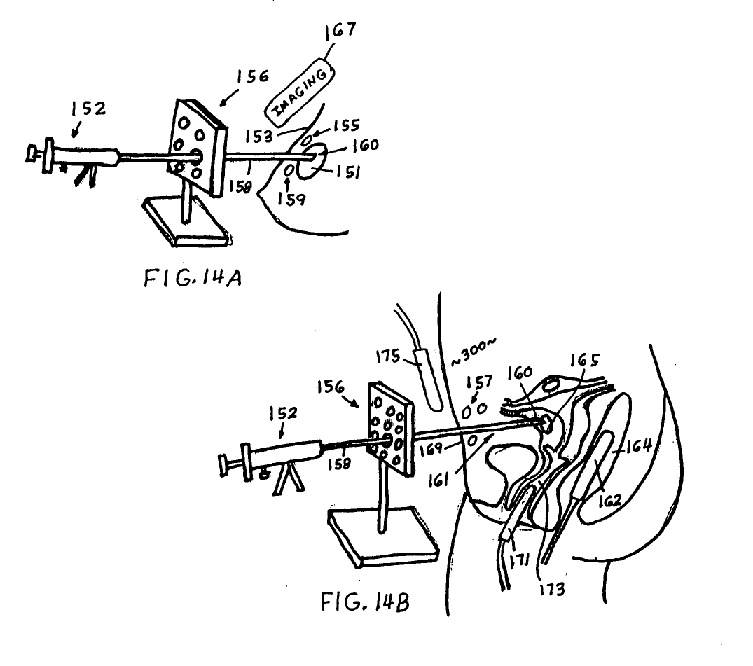

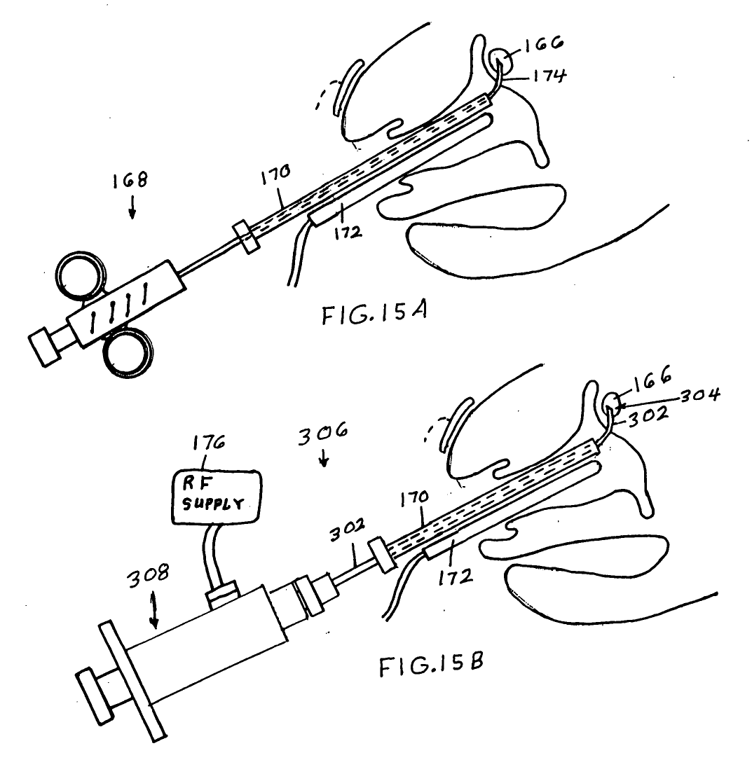

Transvaginal access to a uterine fibroid 166 is illustrated in FIG. 15A with a biopsy device 168 having an introducer tube or needle guide 170, using an ultrasound imaging probe 172, or a transvaginal biopsy probe to aid in guiding a biopsy needle 174 to the fibroid 166. FIG. 15B illustrates using the introducer tube or needle guide 170 for guiding a treatment substance injection needle 302 into the fibroid 166, for example after doing a biopsy procedure illustrated in FIG. 15A and removing the biopsy needle 174. The injection delivery needle i.e. transvaginal injection needle device (22-14 ga size) 302 preferably has an echogenic tip 304 and is designed to inject a treatment substance as described in the above text and figures of the drawing under ultrasound imaging guidance. The needle 302 is percutaneously inserted into the uterus wall and into the target tissue of the fibroid 166. The device 302 and similar devices of other configurations for percutaneous functions will be referred to as a percutaneous device, as well as a transvaginal or transcervical device when used to access the uterus. The injection device needle tip 302 can be straight, curved, angular or articulating to inject any part of the fibroid 166 and/or uterine cavity anatomy. The injection device 306 includes the needle 302 and a treatment substance injector apparatus illustrated symbolically and as one embodiment as a syringe 308. The apparatus 306 can be designed to allow semi-automatic or automatic injection treatment operation. The echogenic injection needle tip and treatment substance are visible as bright white echogenic reflections in imaging observation, which can be controlled by the volumetric dosage of the treatment substance. The injection needle 302 can be inserted into the uterus using other alternate approaches, such as through a working channel of a transvaginal ultrasound probe or through a working channel of an endoscope or hysteroscope. Ultrasound probe devices are illustrated in reference to FIGS. 17 and 18. A typical dosage of treatment substance for treatment of a fibroid varies between 10-30% of its volume as measured by ultrasound imaging. FIG. 15B also shows an optional RF supply 176 for application of RF (radio frequency) energy to the fibroid. The use of RF energy is described in more detail in U.S. patent Ser. No. 09/510,537.

FIG. 16 shows use of a probe apparatus 177 for accessing the uterus 179. The probe includes an endoscopic viewing device 181 for guidance. A treatment substance injection needle 183 is inserted through the probe 177, and can be adjusted by control apparatus 185 as illustrated in reference to FIG. 10. FIG. 16 shows the needle 183 inserted into a uterine fibroid 187. Alternatively, RF energy can also be applied through an RF connector 189. Further details referenced in the present disclosure regarding application of RF energy apply as well as an alternate embodiment to the application illustrated in FIG. 16. Injection of a treatment substance is illustrated by inclusion of syringe 191, and luerlock connector 193 for injection of the treatment substance through the hollow core needle 183. Since the device 177 is used to access the uterus, it is also referred to as a hysteroscope device, and since the needle accesses the target tissue 187 percutaneously, the device 177 is also referred to as a percutaneous device.

The preferred injectable treatment substance used for interstitial injection treatment of breast fibroadenoma, cysts and fibroids consists of one or more selections from a family of chemo-gels and viscous injectable formulations including; ethanol gels, saline gels, biological gels, chemotherapeutic gels, biological toxin gels, neurotoxin gels, bioabsorbable gels, polymer gels, pharmaceutical gels and other proprietary gels and viscous substance formulations. The treatment substance consists of an aqueous, viscous composition of an active treatment substance and an inactive binding (carrier) substance, and may also include other complimentary chemical agents including for example a buffer substance, and/or epinephrine and/or an echogenic contrast agents etc. as required. The inactive binding substance provides appropriate viscosity to the treatment substance as explained above. The composition, molecular weight and concentration of the active treatment substance in relation to other agents and additives can determine the physical and chemical properties of the treatment substance. The inclusion of an echogenic contrast substance causes the treatment substance formulation to be readily visible under ultrasound, CT and MRI imaging. The treatment substance then has a visible characteristic allowing real time, interactive control during injection treatment by varying the dosage volume. The active treatment substance portion of the treatment substance, its concentration, specification and physical properties are designed to create an optimum therapeutic effect in target tissue for treatment of various gynecological and reproductive organ disorders.

Detailed specifications and physical properties for various treatment substance formulations have been established for treatment of breast tumors, cysts, fibroadenomas, fibroids, endometrial ablation, uterine cavity ablation and other gynecological disorders. The treatment substance formulation and optimum injection volume dosage have also been established for treatment of various disease conditions. The detailed formulations for treatment substances are outlined in FIGS. 5A and 5B for treatment of the breast, and FIGS. 6A and 6B for a variety of organs.

The percutaneous and interstitial injection treatment using a treatment substance (illustrated in FIGS. 8 and 9) has potential application for diseases of various body organs including breast, uterus, lungs, liver, kidney, myomas, ovary, fallopian tube, fibroids, rectum, adrenal gland, gallbladder, etc. The controlled tissue ablation in a body organ or body cavity can be accomplished with the method and apparatus described above for treatment of diseases including breast cancer, uterine fibroids, kidney tumors and various other diseases; and for treatment of diseases of the fallopian tube, ovary, lung, liver and other organs; and for performing injection of a treatment substance into any organ for treatment of various diseases, and wherein the treatment substance can be of any formulation, and for causing tissue ablation including for example uterine cavity ablation and endometrial ablation.

The viscous treatment substance, such as a chemo-gel, creates a localized desirable effect in the target tissue without causing undesirable side effects in surrounding body organs or a systemic effect in the entire body. The treatment substance specification for a specific disease treatment includes its composition, % concentration, physical properties including viscosity, molecular weight and specific gravity, along with an appropriate dosage level for an optimum clinical outcome. The general list of various active treatment substances and inactive binding (carrier) substances used for injection treatment are outlined in FIGS. 2, 3 and 4. Furthermore, a specific formulation, specification and dosage level for treatment of each specific disease indication is outlined in FIGS. 6A and 6B for a variety of organs and in FIGS. 5A and 5B for breast diseases. The injectable treatment substance formulations, specifications and dosage levels for treatment of breast diseases including breast tumors, cysts, fibroadenomas and breast cancer; and for uterine cavity and endometrial ablation are listed in FIGS. 5A and 5B.

FIG. 17 shows an ultrasound probe apparatus 178 with an internal needle guide or biopsy channel 182, and FIG. 18 shows an ultrasound probe 188 with external needle guide apparatus (190, 192) for guiding a needle. The apparatus of FIGS. 17 and 18 can be inserted through a natural body passage such as a rectum, vagina, or vagina and cervix, or through an incision for access to an internal body organ. For example, in FIG. 16, the apparatus 177 can be replaced with the apparatus of either FIG. 17 or 18. In this case, the probe and needle apparatus would be called a endocavity ultrasound probe apparatus/device, or a transvaginal ultrasound probe with an external needle guide as in FIG. 18, or an internal needle guide channel as in FIG. 17. Similarly, if the apparatus is used by insertion into the rectum, it would be called a transrectal ultrasound probe device, and if through an incision in the abdomen, it is called a laparoscopic ultrasound probe device. With the apparatus of FIGS. 17 and 18 appropriately dimensioned, it can also be used to access body parts including the urethra, bladder and prostate. In this case, it would be called a cystoscopic or transurethral ultrasound device/apparatus.

Referring specifically now to FIG. 17, a combination needle guide and endocavity ultrasound probe apparatus 178 is shown including a functional ultrasound probe portion 180 for imaging, and a channel 182 built into the probe apparatus 178 for guiding a hollow core needle 184, wherein the needle 184 is configured for injecting a treatment substance as disclosed above.

In operation, the needle 184 is retracted so as to place the tip 187 inside the channel 182. The probe 180 is then inserted into a body passage, such as a rectum. When the operator observes via the ultrasound imaging that the probe is placed as required for insertion of the needle 184 into a target tissue, the needle is thrust forward into the tissue to the desired depth, which can be observed through use of the ultrasound imaging apparatus. The treatment substance is then propelled through the needle 184 by use of a propulsion injection device symbolically illustrated by syringe 185.

FIG. 18 shows a combination needle guide and ultrasound probe apparatus 186, including an ultrasound probe apparatus 188 for imaging, and an attached needle guide apparatus (190, 192) for guiding a hollow core needle 194 along the outside of the probe 188. In commercially available equipment, guide apparatus such as 190 and 192 is provided for guiding a biopsy needle. According to the present invention, this biopsy needle guide apparatus is used to guide the needle 194 configured for injection of a viscous treatment substance. The operation of the apparatus 186 involves first placing a protective covering (condom) over the needle guide and probe assembly, with the needle in a withdrawn position behind the tip 196 of the probe 188. Alternatively, the needle tip can be retracted within a structure such as guide support 190, and thereby also preventing the needle tip from penetrating body tissue while the probe and needle assembly 186 is being positioned within a body passage. The probe and needle apparatus 186 is then inserted into a body passage such as a rectum. With the probe tip 196 in the desired position for inserting the needle 194, the needle 194 is thrust forward, through the protective covering (not shown), and into the target tissue (not shown) to the desired depth, which can be monitored by an ultrasound imaging apparatus including the probe and related instrumentation not shown. The treatment substance is then propelled through the needle 194 by a propulsion/injection device 197 symbolically illustrated as a syringe.

Focusing particularly now on a procedure for chemo-ablation of uterine fibroids, the above descriptions illustrate a variety of methods and apparatus for injecting a viscous treatment substance into a fibroid, and the specific treatment substance formulation has been set forth in the figures of the drawing. The procedure of chemo ablation of a uterine fibroid is preceded by screening against cancer of the affected region. A PAP smear and endometrial biopsy/curettage must exclude cancer or precancerous lesions of the uterus and cervix. If a fibroid uterus is suspected, then an ultrasound examination should be performed to exclude ovarian masses. It is preferable if the patient is post menstrual or has been started on Lupron, or the equivalent which causes shrinkage of fibroids. However, the use of Lupron is not a requirement.

The injection needle/catheter delivery devices of the present invention as described above can administer the viscous treatment substance for chemo ablation of fibroids based on type, size and location in the uterine cavity, using the various instrumentation described in the present disclosure, including hysteroscopic imaging guidance or non-invasive ultrasound imaging guidance.

The size and location of the fibroid can be determined by use of an abdominal ultrasound device or transvaginal ultrasound imaging probe. The delivery of the treatment substance to the target tissue can be carried out via a selected one of the methods described above, including percutaneous, transvaginal, transcervical, hysteroscopic or laparoscopic methods.

Subsequent to insertion of the injection needle device in the fibroid, the position of the needle and a desirable depth in the fibroid can be confirmed by real time ultrasound imaging. The physician can also monitor the chemo-gel in the fibroid tissue in “real time” as it is being injected using an ultrasound imaging probe/device. The chemo-gel can cause controlled tissue ablation of the fibroid under imaging guidance without creating any adverse effect to surrounding organs. The chemo-gel treatment substance, its concentration, composition and formulation and recommended dosage are outlined in FIG. 6A.

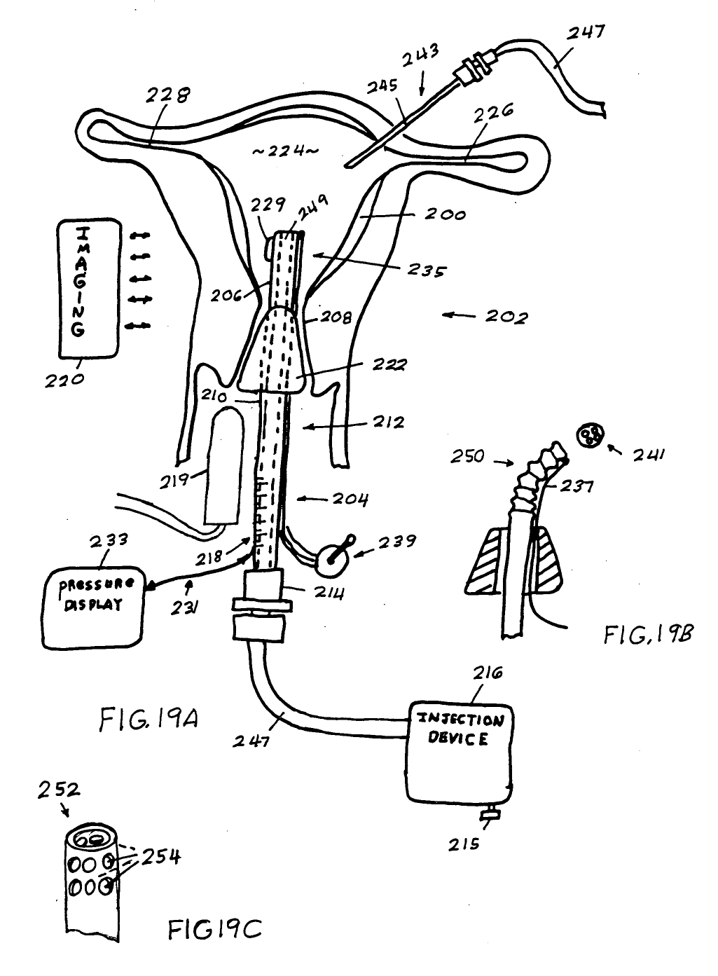

FIGS. 19A and 19B are now referred to for illustrating a method of uterine cavity or endometrial ablation i.e., chemo ablation of the endometrium 200 of uterus 202. The chemo ablation procedure is preceded by screening against cancer of the affected region and physical condition within established norms. A PAP smear and endometrial biopsy/curettage must exclude cancer or precancerous lesions of the uterus and cervix. If a fibroid uterus is present, an ultrasound should exclude ovarian masses. The uterine cavity measurement must be obtained prior to determining a volumetric injection dosage and performing the chemo ablation treatment procedure.

It is preferable if the patient is post menstrual, or has started on Danazol or the equivalent, which causes reduction in bleeding and a thin endometrium, at a rate of 800 ml daily, from the 5^{th} day of the previous menstrual period until two weeks after the procedure. However, the above-mentioned is not a requirement. The patient for example can normally undergo the procedure in an ambulatory surgery unit or outpatient facility where Valium and/or Demerol can be given intravenously for added comfort.

A viscous substance injection device i.e. delivery catheter apparatus 204 is inserted after a bimanual examination and speculum of the cervix. Prior to insertion of the apparatus 204, the physician angulates the catheter 206 of the device by bending it to the desired angle of articulation to accommodate the anatomical structure of the particular patient. Dilation of the cervix 208 to 6 mm may be required which may necessitate a local 1% lidocaine block of the cervix. Once in place the delivery catheter stem 210 protrudes from the vagina 212, terminated at a luer lock connector 214 for attachment of a syringe or injection device/injector (symbolically represented as block 216) filled with the chemo-gel treatment substance.

Accurate placement of the injection needle or delivery catheter apparatus 206 can be facilitated by observing distance markings 218 on the catheter stem 210 indicating depth of insertion and by real time monitoring using non-invasive imaging modalities including ultrasound, CT, MRI and X-Ray, represented by block 220. An ultrasound device for use in this embodiment includes an abdominal ultrasound probe, positioned adjacent the abdominal area. Another ultrasound device is a transvaginal ultrasound imaging probe 219 placed in the vagina as shown. Placement of the catheter 206 can also be guided through use of a miniature endoscope that can be inserted through one of multiple lumens in the catheter. Multiple lumens are shown in end view 241 of FIG. 19B. FIG. 19A also shows a tapered sealing plug 222 through which the catheter 206 passes. The plug 222 is installed for the purpose of sealing the cervix and retaining the injected treatment substance in the uterine cavity 224. Upon placement of the delivery apparatus in proper position, it can be connected to an injection device containing a treatment substance, along with pressure monitoring or image guiding instruments. Alternatively, the cervix can be sealed with an inflatable balloon mounted on an interuterine catheter and positioned inside the uterine cavity or using a double balloon mounted on an intrauterine catheter, positioning them on either side of the cervix. Although FIG. 19A shows a catheter 204 passing through the sealing plug 222 into the cavity 224, the present invention also includes use of other devices for injecting the treatment substance. For example, the catheter 204 in FIG. 19A as shown can be replaced with an endoscopic/hysteroscopic device similar to that shown in FIG. 10 or FIG. 12, having a treatment fluid injection channel for a catheter/hollow core needle, and providing endoscopic visualization of the uterine cavity for positioning the catheter/needle tip. The endoscopic device in this case would have all channels sealed sufficiently by closing the outlet port and by creating a closed system to allow the proper pressure build-up in the uterine cavity.

FIG. 19A also shows a percutaneous injection device 243 including a hollow core needle 245 for percutaneously and interstitially accessing the uterine cavity 224 for injection of the viscous treatment substance as an alternate embodiment. In this case, line 247 from the injection device 216 would be connected as shown to the device 243. The sealing plug 222 and catheter 206 would then simply be replaced with a plug apparatus for retaining the pressure in the cavity, but the sealing plug catheter apparatus could also have a pressure sensor such as 229 with connection to a display 233. Actually, FIG. 19A illustrates this situation if the catheter has a closed tip 249. The absence of a passage/lumen allows the catheter shown to function as a plug, blocking the injected substance through the needle 245 from escaping the uterine cavity.

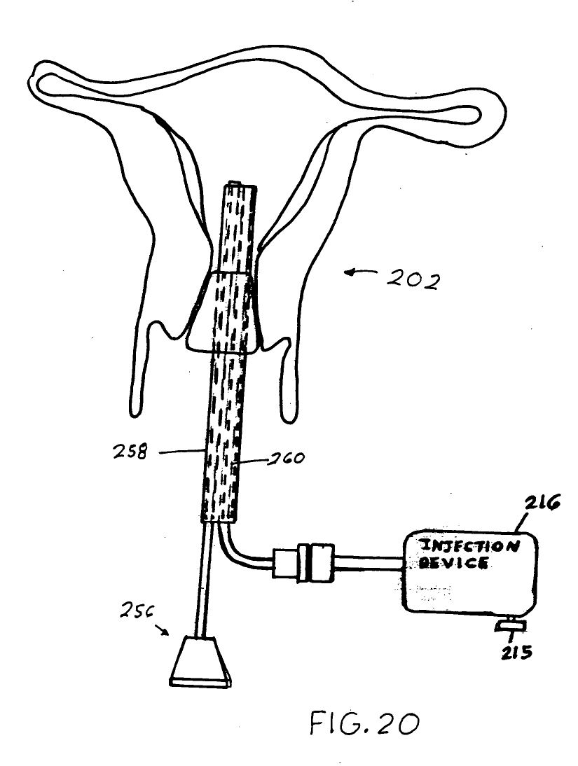

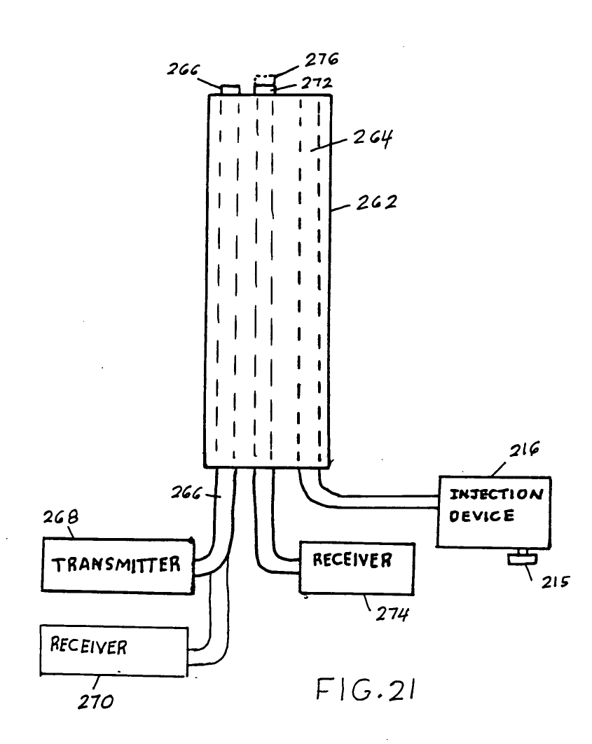

Subsequent to insertion of the delivery catheter apparatus, uterine cavity 224 measurements can be confirmed by real time ultrasound imaging. An optimum intracavity pressure for the chemo-gel can also be measured by infusing into the distensible uterine cavity a non-reacting/neutral gel formulation and increasing the injection pressure and volume of the gel, while observing its distribution in “Real Time” with an imaging method until the gel begins to leak from any areas where the chemo-gel is not desired, specifically the fallopian tubes 226 and 228 to the ovaries, and past the plug 222 outside the cervix 208 to the vaginal canal. The uterine cavity pressure can be measured with a pressure sensor 229 attached to the distal end of the catheter 206 as shown. The pressure sensor 229 is attached to sensor wires 231 for example that can be embedded in the catheter 206 wall and outputting to a sensor display apparatus 233. The pressure sensor and corresponding interconnections and display can be of any type, using pressure sensor technology including fiberoptics, silicon, differential, etc. The catheter apparatus 204 can alternatively also include a mechanism for articulating a segment of the tip 235. The apparatus can be similar to that disclosed in reference to the endoscope 72 in FIG. 10 and further described in U.S. patent Ser. No. 09/510,537 and 09/715,853 incorporated by reference. FIG. 19B shows a catheter tip 250 corrugated or designed as a bellow for facilitating bending and articulating in response to tension applied to a wire 237 applied by any of various means, such as with a device symbolically illustrated as item 239. The catheter 206 can have one or more lumens, for example for allowing insertion of a miniature endoscope. Multiple lumens are illustrated in a catheter end view at 241 in FIG. 19B. An area of the catheter 206 including at least a portion of the length designated as 235 can alternatively be radiopaque or echogenic in order to aid in viewing the placement of the catheter in the uterus using an imaging method. The intrauterine catheter tip 235 can have various holes, configurations and tip profiles, such as a closed tip, a bullet nose tip, or a round tip with one or more holes. FIG. 19C shows a catheter tip 252 that has holes 254. FIG. 20 illustrates the use of a miniature endoscope 256 inserted through a channel of a catheter 258 for viewing the uterine cavity. A catheter channel 260 is shown for injection of the treatment substance. FIG. 21 is an illustration of alternative methods of viewing inside a uterus, and for illustrating a method of tissue recognization. A catheter 262 is shown for use instead of catheter 206 of FIG. 19A or catheter 258 of FIG. 20. Catheter 262 has a lumen 264 for injection of a treatment substance propelled by an injection device 216. One embodiment illustrated in FIG. 21 uses an optical fiber 266 to input light of a specific wavelength from a transmitter 268. The light entering the uterus is reflected by uterine tissue and transmitted back along the fiber 266 to an optical receiver 270. Alternatively, the reflected light can be transmitted along a second optical fiber 272 to receiver 274. The reflected light system described can be used for tissue analysis and recognization by transmitting a specific wavelength of light and analyzing the reflected light wavelength received by receiver 274 or 270. The color change between the two wavelengths and shift in the wavelength can be processed to identify the tissue characteristics and composition, therefore defining a tissue recognition system.

FIG. 21 also illustrates an embodiment wherein light can be transmitted to the uterus by way of fiber 266, and detected by a charge coupled device (CCD) 276. The detected output from the CCD is sent to a receiver along a conductive path represented by 272 to a receiver, again illustrated by item number 274. Once the “leakage pressure” limit is established in the uterine cavity using a neutral gel or viscous substance, sufficient care should be taken not to exceed this pre-set pressure limit during injection of the chemo-gel for the endometrial ablation procedure. The uterine cavity pressure can be monitored in “real time” basis using a pressure sensor during injection treatment. The neutral gel substance is then evacuated and thoroughly rinsed from the uterus.

The physician can then slowly inject the chemo-gel into the uterine cavity until a pressure gauge display 233 indicates that the fluid pressure is within the preset constraints. The volume required to fill the cavity 224 is based on uterine cavity measurements and should not exceed a pre-set volume determined by ultrasound imaging. The position of the chemo-gel is also confirmed with real time ultrasound imaging during the injection procedure.

Upon completion of chemo ablation, a pressure valve 215 on the injection device 216 is released to allow the chemo-gel to be withdrawn from the uterine cavity 224, causing the uterus to deflate or collapse. Upon deflation of the uterine cavity, the delivery catheter apparatus 204 may be safely withdrawn from the patient. The uterine cavity must then be rinsed and aspirated thoroughly to remove residual chemo-gel from the uterine cavity 224.

The intrauterine catheter of the present invention may be made of the following materials: ABS polymer, PEBAX polymer, polycarbonate, HYTREL polymer, C-FLEX polymer, or any conventional biocompatible polymeric material having sufficient rigidity and/or flexibility to effectively provide the desired insertion and use properties, and equivalents thereof.

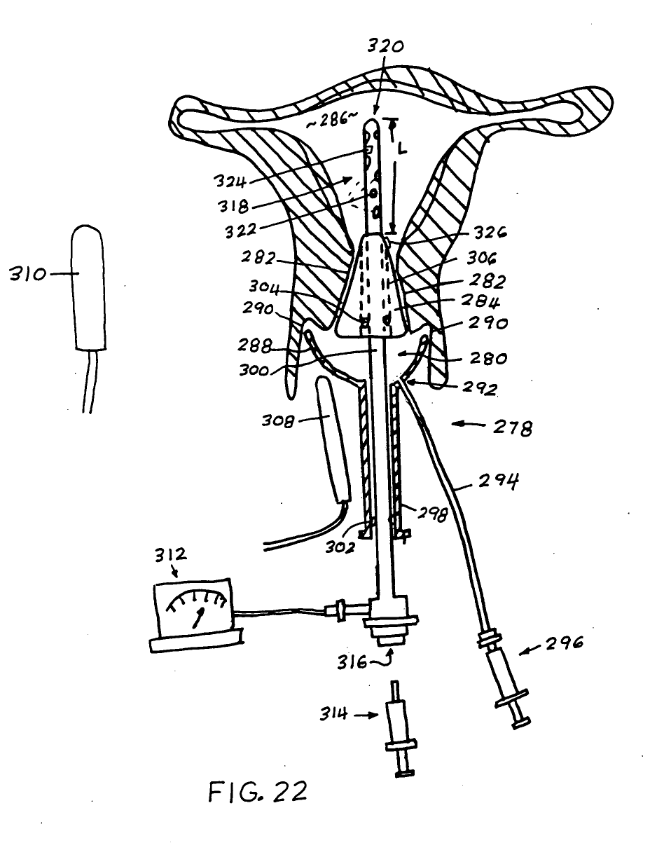

FIG. 22 illustrates an apparatus 278 for providing a degree of vacuum to the cervical area in the vagina canal 280 and for the purpose of causing the external (atmospheric) pressure to force and thereby seal the cervix 282 against the plug 284 in order to retain an injected treatment substance in the uterine cavity 286. The apparatus 278 includes a cup shaped device 288 for sealing against tissue 290 of the vagina and/or cervix. A vacuum port 292 leads via a vacuum line 294 to a vacuum pump 296 illustrated symbolically as a hand held vacuum type of device similar to a syringe, but which can be any kind of vacuum pump apparatus designed so as to be capable of providing the required vacuum. The apparatus 278 has a tube 298 extending from the cup device 288, through which a catheter 300 can pass. The tube 298 and/or catheter 300 in one embodiment are of dimensions so as to provide an adequate vacuum seal between them to retain a sufficient vacuum in the vaginal area 280 in the cup device 288. Alternatively, an O-ring 302 can be used to achieve the necessary seal. Similarly, an O-ring 304 can be placed between the catheter 300 and the plug base 306 for providing the required seal to retain the vacuum in the vaginal cavity 280, and the treatment substance in the uterine cavity 286. Alternatively, the sealing plug can be designed from rubber, silicone, kryton or other materials with self sealing properties. The length “L” of the catheter 300 in the uterine cavity 280 is adjustable by sliding the catheter 300 through the plug 284 and outer tube 298. The adjustability of the catheter, and the seal between the catheter and plug applies also to the configurations in FIGS. 19A, 20 and 21. Alternatively, the catheter 300 can be non-adjustably installed through the plug 284, or as a further alternate embodiment as an integral part of the plug 284, and these alternates also apply to FIGS. 19A, 20 and 21. Alternatively, the cervix can also be sealed using a delivery catheter with single or double balloons, inflated to a proper size to prevent any leakage of injected substance from the uterine cavity. For purposes of illustration, the plug 222 can represent a balloon, which can also, or alternatively be on either the input and/or output of the cervix.

FIG. 22 also shows an ultrasound probe 308 installed in the vaginal cavity, and an ultrasound probe 310 symbolically placed to represent an ultrasound probe in the abdominal area. Either of these or an ultrasound probe in the rectum, as illustrated in the above figures can be used in an ultrasound imaging system as explained above for guiding placement of the catheter 300, and alternatively for observing and monitoring a viscous treatment substance in the uterine cavity 286 and adjoining areas including the fallopian tubes to avoid possible adverse effects. FIG. 22 also shows a pressure monitor 312 connected through the catheter to the cavity 286 to measure the treatment substance pressure in the uterine cavity 286. Alternatively, a pressure transducer 324 can be incorporated into the catheter tip 320, for example on the top 318, or on the inner end 326 of the plug 222, with an electrical connection to transmit pressure data to a meter, represented as item 312. A syringe 314 is shown, representing apparatus for supplying/injecting the substance into the catheter 300 through an input 316.

The tip or i.e. distal end length 318 of the catheter can include one or more openings for a substance to enter the cavity 286. The tip can be any of various shapes, including for example round or bullet shaped. The end 320 can be either open, or closed with one or more holes 322 for exit of a substance into the cavity 286. The holes 322 can have any profile, configuration or arrangement to provide optimum diffusion of a substance according to the present invention with any particular viscosity.

In summary, the present invention relates generally to methods and apparatus for injection treatment of various injectable treatment substances. The present invention includes the substance concentrations, compositions, formulation and other physical properties of the treatment substances to achieve optimum parameters for treatment of body organs including the breast, fibroids and uterus, for achieving endometrial ablation and for treatment of diseases associated with other female reproductive organs. The injection treatment substance is injected into a breast or uterus or other body, organ in the form of a gel or highly viscous substance for a controlled therapeutic or tissue effect. The viscous chemo-gel formulation of the injectable treatment substance creates a localized tissue effect in the target area without causing undesirable side effects in surrounding organs or throughout the patient's body. The chemo-gel formulation is injected into a diseased body portion through use of any one of various devices known to those skilled in the art. This was illustrated in FIG. 8 figuratively illustrating injection devices 50, which can be applied to any organ as required. A laparoscope or endoscope device, known to those skilled in the art, can be inserted through an incision for use in guiding an injection needle to a target tissue, such as the liver, kidney, uterus, bladder, breast or lung, or other organ. In guiding a needle to a precise target, the viewing endoscope or laparoscope or other similar device is often helpful. The use of a non-invasive ultrasound imaging technique is also included in the spirit of the present invention for guiding a needle. This is helpful in guiding a biopsy device, and can also be used as additional guidance when using an endoscope or similar device.

Although the present invention has been described above in terms of a specific embodiment, it is anticipated that alterations and modifications thereof will no doubt become apparent to those skilled in the art. It is therefore intended that the following claims be interpreted as covering all such alterations and modifications as fall within the true spirit and scope of the invention.

1-24. (canceled)

25. A method of treatment, comprising:
a) measuring the volume of a body cavity;
b) inserting an apparatus into the body cavity;
c) delivering a treatment substance into the body cavity, wherein said treatment substance includes an active treatment substance and an inactive binding substance.

26. The method of in claim 25, wherein the treatment substance is further formulated so as to be visible in real time using a non-invasive imaging method.

27. The method of claim 25, wherein the body cavity is a uterus, uterine cavity, fallopian tube, vagina, or cervix.

28. The method of claim 27, further comprising creating a vacuum, relative to ambient pressure, around a cervical area.

29. The method of claim 25, wherein the inactive binding substance includes one or more gelling agents including one or more polymers.

30. The method of claim 25, wherein the active treatment substance is selected from the group consisting of ethyl alcohol, hypertonic saline, saline, acetic acid, enzymes, epinephrine, biological toxins, chemotherapeutic agents, pharmaceutical drugs, and natural extracts.

31. The method of claim 30, wherein the active treatment substance is ethyl alcohol in an amount equal to 70 to 99.0 percent of said treatment substance.

32. The method of claim 30, wherein the active treatment substance is saline or hypertonic saline in an amount equal to about 20 to 40 percent of said treatment substance.

33. The method of claim 25, wherein inserting an apparatus comprises a method selected from the group consisting of percutaneous and interstitial insertion, insertion through natural body openings including vagina and rectum, insertion through a guide channel of an ultrasound imaging apparatus, insertion through an endoscope needle guide channel, insertion through a channel in a biopsy needle apparatus, insertion through a channel in a laparoscope, insertion through a channel in an endoscopic hysteroscope, insertion through a channel in a cytoscope, insertion through a channel in a falloposcope, and insertion through a channel of a resectoscope.

34. The method of claim 25, wherein the apparatus is guided to a target location using an instrument selected from the group consisting of ultrasound imaging apparatus, CT, MRI, and X-Ray imaging apparatus, an endoscope, a biopsy device, a laparoscope, an endoscopic hysteroscope, a cytoscope, a falloposcope, and a resectoscope.

35. The method of claim 25, wherein the treatment substance includes at least one physical form of material selected from the group consisting of liquids, gases, solids, gels, viscous fluids, semi-liquid solutions, semi-solids, suspensions, colloids, foams, composites, micro-spheres, pallets, and conjugates.

36. The method of claim 25, wherein measuring the volume of a body cavity is conducted using real time ultrasound imaging.

37. The method of claim 25, wherein the treatment substance is formulated to be an electromagnetic energy conductive substance to enhance a tissue ablation effect when used in conjunction with application to said target tissue of an energy selected from the group consisting of microwave, laser, ultrasound, magnetic, ultraviolet, infrared, visible, x-ray, and combinations thereof.

38. The method of claim 25, wherein the apparatus comprises an echogenic tip.

39. The method of claim 25, wherein the treatment substance is designed and formulated for treatment of a specific body part and disease or disorder.

40. The method of claim 39, wherein the disease or disorder is selected from the group consisting of fibroadenoma, uterine fibroids, myomas, polyps, excessive uterine bleeding, uterine cancer, fallopian tube disorders, ovarian tumors and cysts, ovarian cancer, cervical cancer, vaginal warts, and condolomas.

41. The method of claim 39, wherein the treatment substance is for a treatment selected from the group consisting of fallopian tube ablation, ostium ablation, contraception, fibroid and polyp ablation, and endometrial ablation of a uterine cavity.

42. The method of claim 25, further comprising aspirating the treatment substance.

43. The method of claim 42, further comprising inspecting the body cavity after aspirating the treatment substance.

44. The method of claim 25, further comprising inspecting the body cavity after delivering a treatment substance.
